(19) United States
(12) Patent Application Publication
(10) Pub. No.: US 2018/0126136 A1
(43) Pub. Date: May 10, 2018

(54) HAND-HELD FLUID-SUBSTANCE APPLICATOR

(71) Applicant: MATBLOK VENTURES INC., Calgary (CA)

(72) Inventors: Steven Tyler BROWN, Calgary (CA); Jeremy Joshua FARROW, Calgary (CA)

(21) Appl. No.: 15/808,144

(22) Filed: Nov. 9, 2017

Related U.S. Application Data
(60) Provisional application No. 62/420,275, filed on Nov. 10, 2016.

(51) Int. Cl. A61M 35/00

(52) U.S. Cl. CPC A61M 35/003; A61M 2202/04

(57) ABSTRACT

A hand-held applicator for applying a fluid substance to a skin surface is disclosed. The applicator may be wedge-shaped to facilitate the application of the fluid substance to both broad and constricted portions of the user's skin by way of a broad base portion and a narrow end tip portion, respectively. The applicator may also comprise a means for limiting the flow of the fluid substance to the broad base portion of the wedge to facilitate application primarily from the narrow tip portion. The applicator may comprise a means for discharging the fluid substance from the applicator in the form of a user-actuated ratchet member or turning wheel mechanism interconnected to a press plate. The applicator may comprise a handle configured to store in a closed position and to rotate to an open position to enable the application of the fluid substance to hard to reach areas of a skin surface.

### CROSS-REFERENCE TO RELATED APPLICATION

This application claims the benefit of U.S. Provisional Patent Application Ser. No. 62/420,275 filed on Nov. 10, 2016. The contents of the aforementioned application are incorporated by reference herein.

### FIELD

This disclosure generally relates to the field of applicators for dispensing a fluid substance. Such applicators may be employed in personal hygiene/cosmetic uses. More particularly, this disclosure relates to a hand-held applicator for applying a fluid substance, such as for example sunscreen and/or insect repellant, to the skin surface of a person.

### BACKGROUND

Applicators for applying fluid substances to a person's skin surface are principally used in the personal hygiene/cosmetics area. Hand-held applicators for lotions, such as for example sunscreen and/or insect repellant, are gaining popularity as they are designed to reduce the inefficiencies and mess associated with applying lotions by hand that are obtained from a container. Further, the demand for convenient means with which to apply topical skin products is growing in line with the apparent increasing rates of skin cancer. While such hand-held applicators are known in a variety of shapes and sizes, applicators are desired which are suitably sized/shaped for applying lotion to both broad/larger surface areas of skin as well as to smaller/constricted surface areas of skin (e.g., both to a person's shoulders and to a person's nose/behind a person's ears, respectively). Moreover, known mechanisms employed to discharge lotion from conventional applicators have drawbacks. Applicators which can be selectively configured for use on both broad/large areas of skin surface and on narrower/smaller/constricted areas of skin surface are also desired as are applicators which are configurable between compact forms and further reaching/extendable forms.

Additionally, some known hand applicators are generally not particularly well configured for holding when a user's hands are greasy from for example being covered with sunscreen. Some known applicators require the use of two hands simultaneously to operate, which can be problematic in some situations.

Additionally, some known hand applicators are particularly susceptible to having a user's hand(s) that is(are) holding the applicator covered with some of the fluid substance particularly during dispensing of the fluid substance.

Some applicators for dispensing fluid substances, such as for example sunscreen/insect repellant, utilize a container that holds a fluid substance and a propellant to emit an aerosol spray. However, the aerosol spray is not well suited for applying the fluid substance to small/constricted/narrow areas of a person's skin surface. Aerosol dispersed fluid substances tend to be dispersed over a relatively wide area.

Some other known types of applicators for dispensing a fluid substance utilize a roller ball mechanism. However, such devices are prone to leaving visible streaks/tracks of the fluid substance on a person's skin. Additionally, such devices are not well suited to applying a fluid substance to a broad surface area in a reasonable time frame—due to the relatively small applicator surface area that contact's a person's skin surface.

### SUMMARY

In one embodiment, the present disclosure relates to a hand-held applicator for applying a fluid substance to a skin surface, said applicator comprising: a first chamber defining at least in part a cavity for holding a fluid substance, the first chamber comprising an interface surface with a plurality of openings there through; a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a pressure increasing mechanism operable to be actuated by an operator to exert a positive pressure on the fluid substance held within the cavity to cause at least a portion of the fluid substance to move through said openings of said interface surface into said pad; wherein the pad has a top view outer perimeter profile that is generally wedge shaped.

In another embodiment, the present disclosure relates to a hand-held applicator for applying a fluid substance to a skin surface, said applicator comprising: a first chamber defining at least in part a cavity for holding a fluid substance, the first chamber comprising an upper wall providing an interface surface having a plurality of openings there through; a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a force application mechanism operable to be actuated by an operator to exert a force on the fluid substance held within the cavity to cause at least a portion of the fluid substance to move through said openings of said interface surface into said pad from where an operator can transfer a portion of said fluid substance to a skin surface; wherein the pad has first portion and a second portion, and wherein a top view outer perimeter profile of said first portion is generally wider than the top view outer perimeter profile of said second portion, said second portion being adapted for applying said fluid substance to a narrow or small area of a skin surface.

In another embodiment, the present disclosure relates to a hand-held applicator for applying a fluid substance to a skin surface, said applicator comprising: a first chamber defining at least in part a cavity for holding a fluid substance, the first chamber comprising an upper wall providing an interface surface, said upper wall having a plurality of openings there through at said interface surface; a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a pressure increasing mechanism operable to be actuated by an operator to exert a positive pressure on the fluid substance held within the cavity to cause at least a portion of the fluid substance to move through said openings of said interface surface into said pad; said pressure increasing mechanism comprising: a movable plate device located within said cavity of said first chamber and operable to move therein; a movement mechanism generally positioned proximate to said first chamber and operably engaged with said moveable plate device to move said movable plate device within said cavity; said movement mechanism comprising: (i) a rotational member interconnected to said movable plate device, such that rotation of said rotational member causes said movable plate device to move within said cavity of said first chamber; (ii) a gear mechanism interconnected to said rotational member operable such that rotation of said gear mechanism causes said rotational member to rotate; and (iii) a gear drive mechanism operable to be actuated by an operator to drive rotation of said gear mechanism; wherein said gear drive mechanism is operable to be actuated by an operator to cause said movable plate device to move within said cavity to exert a positive pressure on said fluid substance within said cavity to cause at least a portion of the fluid substance to move through said openings of said interface surface into said pad.

In another embodiment, the present disclosure relates to a hand-held applicator for applying a fluid substance to a skin surface, said applicator comprising: a first chamber defining at least in part a cavity for holding a fluid substance, the first chamber comprising an upper wall providing an interface surface with a plurality of openings; a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a pressure increasing mechanism operable to be actuated by an operator to exert a positive pressure on the fluid substance held within the cavity to cause at least a portion of the fluid substance to move through said openings of said interface surface into said pad; said pressure increasing mechanism comprising: (a) a movable plate device located within said cavity of said first chamber and operable to move therein, said movable plate device having a threaded opening there through; (b) a movement mechanism positioned generally proximate to said first chamber and operably engaged with said moveable plate device to move said movable plate device within said cavity; said movement mechanism comprising: (i) a threaded member operably interconnected to said movable plate device, such that rotation of said rotational member causes said movable plate device to move within said cavity of said first chamber; (ii) a turning wheel operably interconnected to said threaded member operable such that rotation of said turning wheel causes said threaded member to rotate; wherein said turning wheel is operable to be actuated by an operator to cause said threaded member to rotate to cause said movable plate device to move within said cavity to exert a positive pressure on said fluid substance within said cavity to cause at least a portion of the fluid substance to move through said openings of said interface surface into said pad.

In another embodiment, the present disclosure relates to a hand-held applicator for applying a fluid substance to a skin surface, said applicator comprising: a first chamber defining at least in part a cavity for holding a fluid substance, the first chamber comprising a wall with an interface surface and having a plurality of openings there through; a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a handle configured for holding by an operator, said handle being movable between an open position and a closed position. In another embodiment, the present disclosure relates to a hand-held applicator for applying a fluid substance to a skin surface, said applicator comprising: a first chamber defining at least in part a cavity for holding a fluid substance, the first chamber comprising a wall with an interface surface and having a plurality of openings there through; a flow restrictor plate having a plurality of openings there through, said restrictor plate being positioned adjacent to said interface surface of said wall of said first chamber; a pad having a surface for contacting a skin surface, said pad being positioned proximate said interface surface of said wall of said first chamber and said pad being fluidly interconnected to said cavity of said first chamber such that at least a portion of said fluid substance within said cavity of said first chamber can be fluidly communicated from said first chamber through the plurality of openings of the interface surface into said pad; a pressure increasing mechanism operable to be actuated by an operator to exert a positive pressure on the fluid substance held within the cavity to cause at least a portion of the fluid substance to move through said openings of said interface surface into said pad; and wherein the flow restrictor plate is configured and operable for actuation by an operator to be movable between: (a) a first position in which a group comprising one or more openings of the interface surface are unblocked which permits the fluid substance to flow through the first group of openings of said interface surface into said pad, and (b) a second position in which at least some of the openings in said group of openings of the interface surface is blocked to restrict the fluid substance from flowing through any or all of the first group of openings of said interface surface into said pad.

Other aspects and features will become apparent, to those ordinarily skilled in the art, upon review of the following description of the specific illustrative embodiments.

### BRIEF DESCRIPTION OF THE DRAWINGS

FIGS. 1A-1F show wedge-shaped embodiments of an applicator in top plan profile view.

FIGS. 1G-1M show alternately shaped embodiments of applicators in a top plan profile view.

FIG. 2A is a partially exploded perspective view of an applicator which has a top plan profile view generally as shown in FIG. 1A. FIGS. 2B and 2C each show a cross sectional view at section lines A-A of FIG. 2A, with the applicator assembled.

FIG. 3A is an exploded perspective view of some components of an applicator according to an embodiment which features a operator-actuated ratchet movement mechanism.

FIGS. 3B and 3C are partially exploded perspective views of the embodiment of FIG. 3A with additional components included.

FIG. 3D is a perspective view of the underside of a chamber cover of the applicator components of FIGS. 3A-C.

FIG. 4A is an exploded perspective view of some components of an applicator according to an embodiment which features a turning wheel movement mechanism.

FIG. 4B is a partially exploded perspective view showing the applicator of FIG. 4A with the turning wheel positioned in place.

FIG. 4C is a perspective view of the underside of a chamber cover of the applicator components of FIGS. 4A-B.

FIG. 5 is an exploded perspective view of some components of an applicator which has an electric motor drive mechanism.

FIG. 6A is a partially exploded perspective view of an applicator according to an embodiment which includes a flow restrictor plate.

FIGS. 6B and 6C show the embodiment of FIG. 6A in top transparent plan view with a flow restrictor plate in the closed and open positions, respectively.

FIG. 6D shows the embodiment of FIGS. 6A-6C in top transparent plan view with side guide rails for the restrictor plate.

FIG. 6E is a lower perspective view of the first chamber of the applicator of FIG. 6D.

FIGS. 6F and 6G are top views of an alternate embodiment of the applicator.

FIGS. 7A and 7B show perspective views of an applicator according to an embodiment with a rotatable extension handle in the closed and open positions, respectively.

FIGS. 7C and 7D show related embodiments which feature means for reversibly locking the handle in the open/closed positions.

FIG. 8 is a perspective view of an embodiment of an applicator with a cap in a closed position covering the pad.

### DETAILED DESCRIPTION

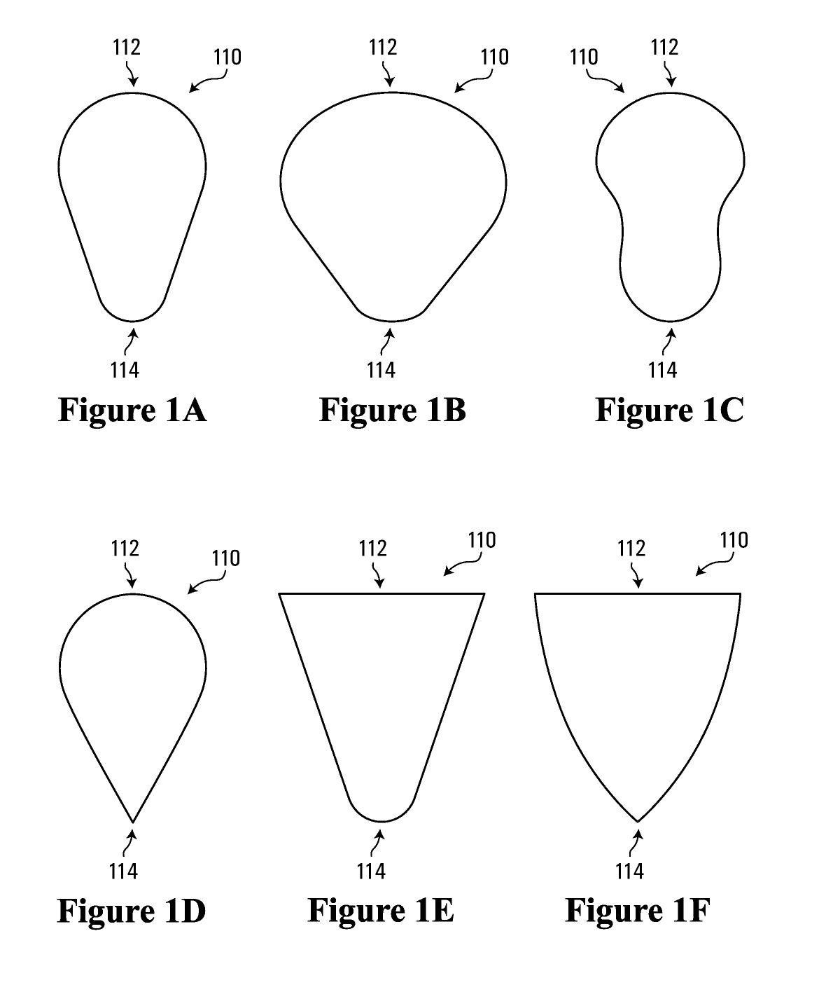

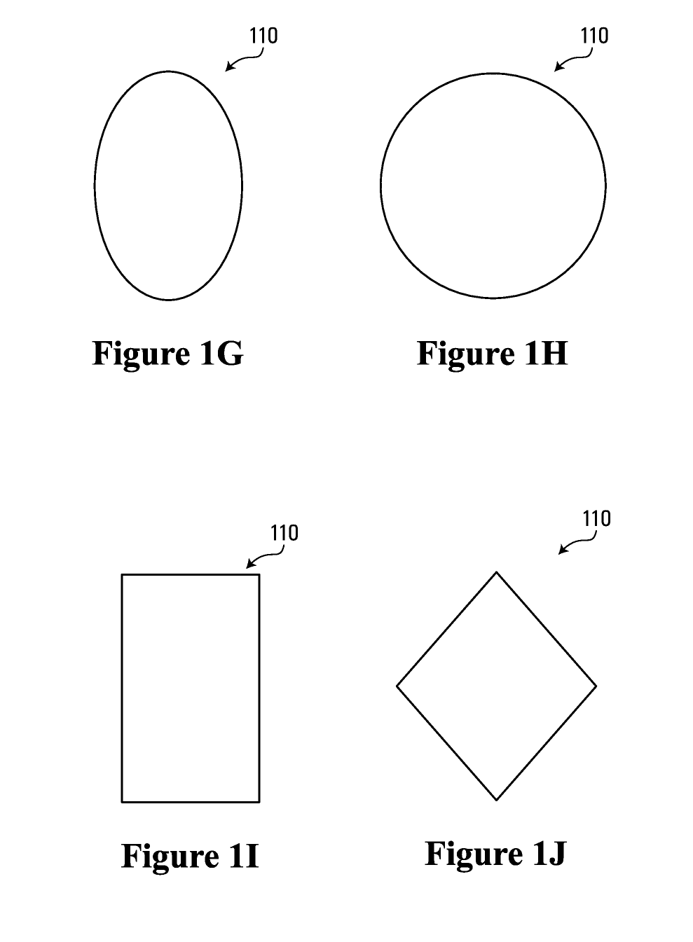

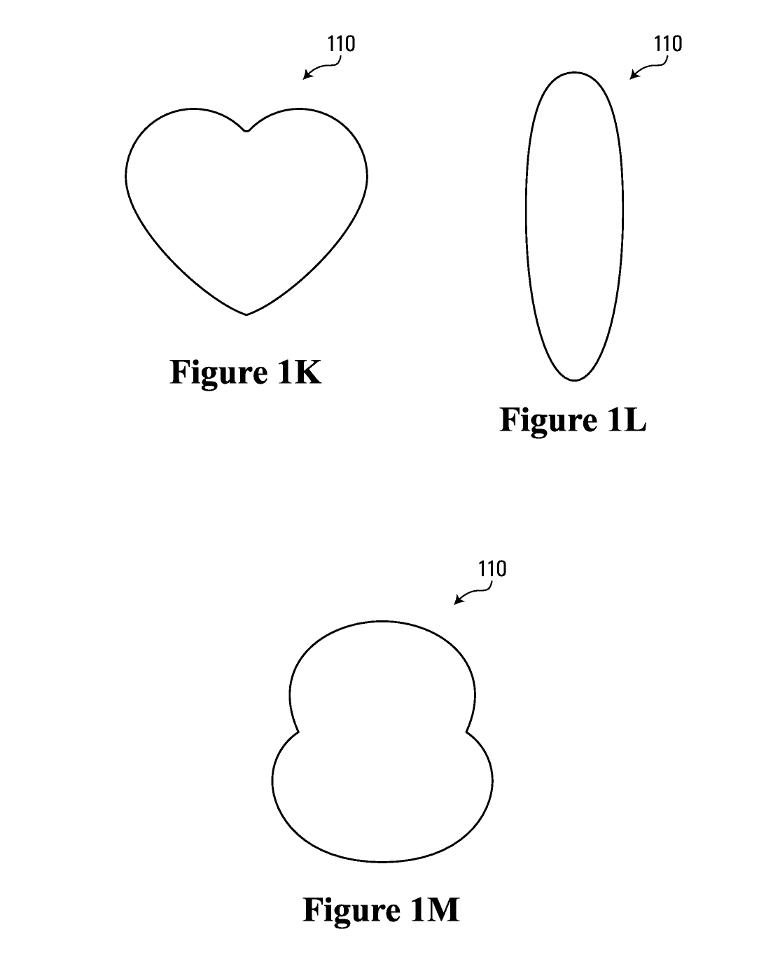

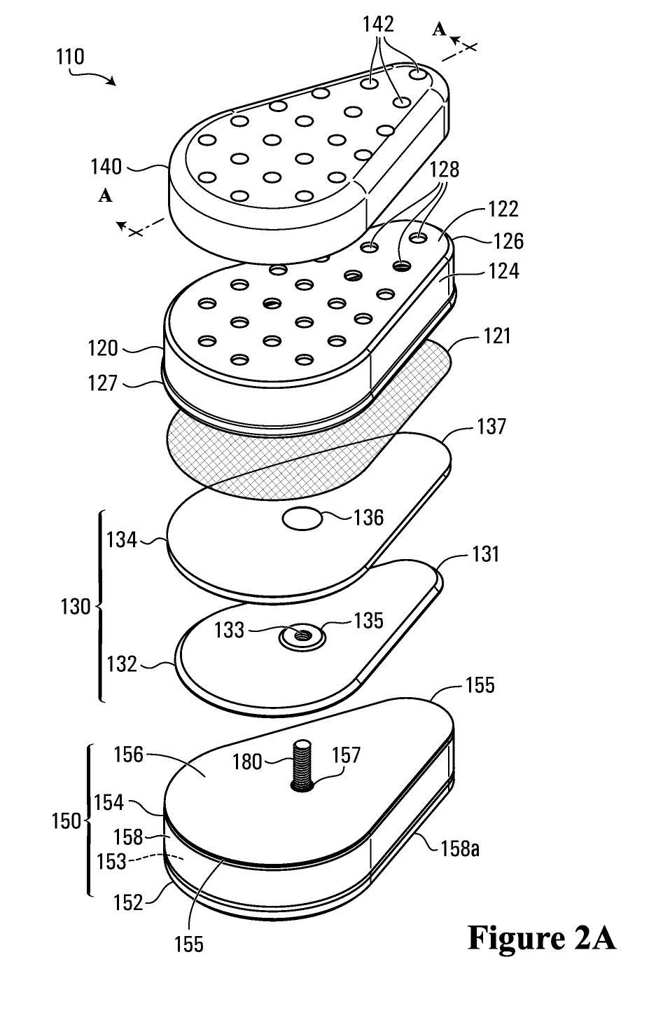

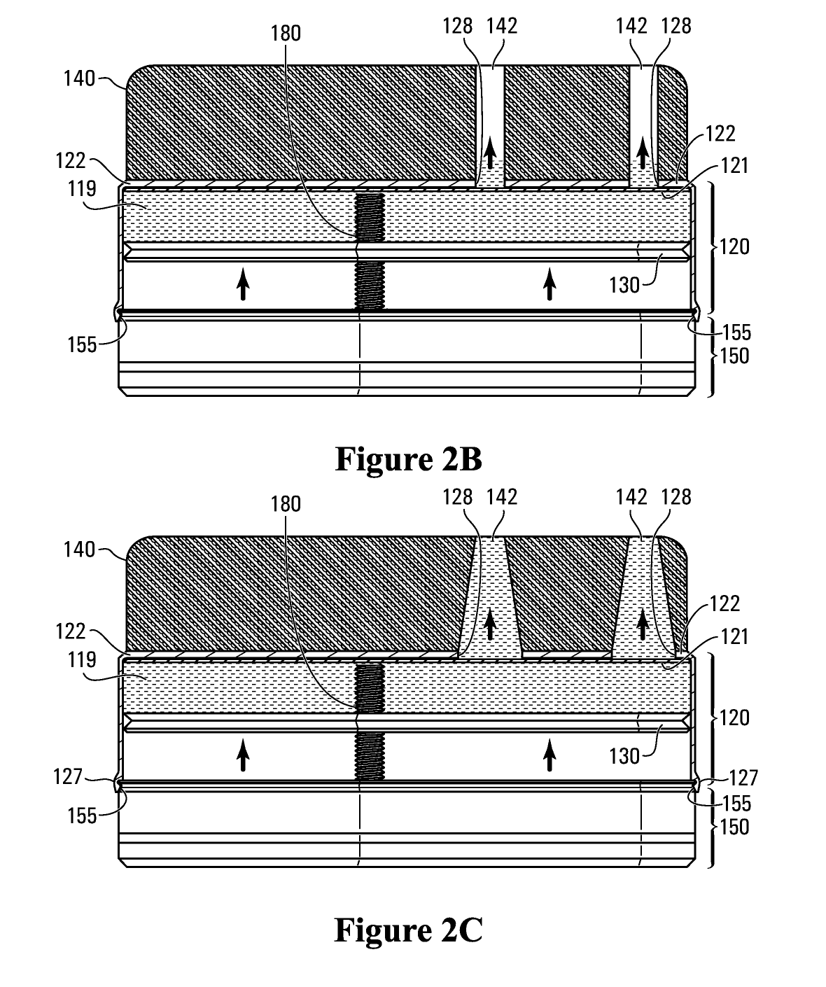

Referring to FIG. 1A, there is shown the top plan view of the general outer perimeter profile for a generally wedge-shaped illustrative embodiment of an applicator 110 for applying a fluid substance 119 (the fluid substance is shown in FIGS. 2B and 2C) to a skin surface. Fluid substance 119 may be any fluid substance suitable for applying to an individual's skin surface of their body, including sunscreen lotion and/or insect repellant.

Applicator 110 may be sized and shaped to be operable with one hand by an operator of the applicator. Wedge-shaped embodiments of applicator 110 may have a portion that generally narrows and may comprise a broad in width, base portion 112 and a relatively narrower end tip portion 114 having a relatively narrower width. Base portion 112, optionally and typically in combination with end tip portion 114, allows for the application of fluid substance 119 to broad portions of a person's skin surface such as the chest, shoulders, back, arms and legs. Having a narrower tip portion 114 allows for the application of fluid substance 119 by applicator 110 to small and/or constricted areas of a person's skin such as the nose and ears. The corners/corner regions/end areas of both base portion 112 and tip portion 114 may be rounded or pointed. Some other top plan view profiles of alternate generally wedge-shaped configurations which may be employed in some other illustrative embodiments are shown in FIGS. 1B-1F, 1J and 1K. While the embodiments of FIGS. 1A-1F are generally wedge shaped in their top plan view general outer perimeter profile, other illustrative embodiments are possible. For example, in some embodiments, applicator 110 may be oval-shaped, square-shaped, circular-shaped or rectangular-shaped, in a top plan view of their general outer perimeter profile (see for example FIGS. 1G-1M). Other shapes which have one or more specifically shaped area portions adapted for applying lotion to particular parts of a person's body are also possible.

Referring to FIG. 2A, there is shown a partially exploded perspective view of an embodiment of an applicator 110 having a top plan view outer perimeter profile generally as shown in FIG. 1A. In this embodiment, applicator 110 comprises a first chamber 120 having an interior cavity configured for receiving and holding a fluid substance 119 such as sun screen lotion and/or insect repellant lotion (not shown in FIG. 2A). First chamber 120 may be made from one or more suitable materials such as for example aluminum or a suitable plastic (e.g. polypropylene or high density polyethylene). First chamber 120 may comprise an upper wall/plate 122 with an edge 126 and a continuous sidewall 124 extending perpendicularly vertically therefrom. Sidewall 124 may include a lower perimeter edge portion 127 that may have a recess.

In one embodiment, plate 122 (and applicator 110 as a whole) has an approximate length dimension in the range of about 10-14 cm, an approximate width dimension in the range of about 6-10 cm at its broadest point, and an approximate width dimension in the range of about 1-5 cm at an end portion. In one embodiment, applicator 110 as a whole has an approximate total depth in the range of about 5-8 cm. In a particular embodiment, plate 122 (and applicator 110 as a whole) has an approximate length dimension of 12 cm, an approximate width dimension of 8 cm at its broadest point, and an approximate width dimension of 3 cm at an end portion. In a particular embodiment, applicator 110 as a whole has a total depth of about 6.5 cm.

Plate 122 may have an array of openings 128 which are suitably sized and spaced to allow fluid substance 119 to pass through plate 122 with the application of a reasonable amount of positive pressure to force fluid substance 119 into pad 140. The size and spacing of the openings 128 may be selected dependent at least in part on the viscosity characteristics of the specific fluid substance 119 that is held in chamber 120 to be dispensed, at typical expected operating temperatures, such that fluid substance 119 only passes through plate 122 and into a pad 140 (as described below) when placed under additional user activated positive pressure/force resulting from the movement of press plate assembly 130 within first chamber 120 towards the openings 128 of plate 122. The array of openings 128 may, for example, include 20 circular openings, relatively evenly spaced from each other on centers of approximately 15 mm with each being approximately 7 mm in diameter. In another embodiment, in which plate 122 has similar dimensions, the array of openings 128 may include in the range of 1-100 circular openings, each having a diameter approximately in the range of 1-50 mm. In some example embodiments, openings 128 may be generally circular in shape, while in other example embodiments, the openings of the array of openings 128 may be of a different configuration such as elongated, narrow slits.

In some example embodiments, first chamber 120 may further comprise a mesh screen 121 with a large number of very small openings that are positioned very close to one another so as to create a mesh. When applicator 110 is assembled, mesh screen 121 may be positioned adjacent and against the inward facing surface of plate 122 within the interior cavity of first chamber 120. Thus positive pressure of fluid substance 119 towards plate 122 in the interior cavity of first chamber 120 may hold the mesh screen 121 in a substantially fixed position against the inward facing surface of plate 122. Mesh screen 121 may also or alternately be held in place by affixing mesh screen 121 to either the inward or outward surface area of plate 122 such as with adhesive bonding.

Mesh screen 121 may be made from one or more suitable materials such as by way of for example nylon, steel wire or a suitable plastic material. Mesh screen 121 may be employed in embodiments where fluid substance 119 is not sufficiently viscous to be held within first chamber 120 by the combination of pad 140 and plate 122 alone when no significant additional pressure is being applied to the fluid substance 119 in first chamber 120 by press plate assembly 130 (as described below). Mesh screen 121 may be sized, configured and oriented to interface with plate 122 and to be bordered by continuous sidewall 124. Screen 121, plate 122 and pad 140 may be configured to co-operate to retain fluid substance 119 in first chamber 120 when applicator 110 is not in use (i.e. when no increase in pressure of the fluid substance 119 is being caused by movement of press plate assembly 130 within the cavity of first chamber 120). The permeability characteristics of mesh screen 121 may be selected based at least in part on the viscosity characteristics of the specific fluid substance 119 that is held in chamber 120 to be dispensed, at typical expected operating temperatures, such that fluid substance 119 only passes through mesh screen 121 and plate 122 and into pad 140 when placed under additional user activated positive pressure resulting from the movement of press plate assembly 130. For example, for use with sun screen lotion, mesh screen 121 may have a mesh size in the range of 8 to 50 mesh. In another example, a 12 plastic mesh for screen 121 may alternatively be utilized.

Applicator 110 may also comprise pad 140 which may be sized, dimensioned, and oriented to interface with plate 122 with a substantially common outer perimeter profile. Pad 140 is for interfacing on one side surface with a person's skin surface (such as the operator's skin surface or the skin surface of a third party) during the application of fluid substance 119. The opposite side surface of pad 140 interfaces with the outwardly directed interface surface of plate 122.

Pad 140 may be formed of one or more materials having generally sponge-like characteristics such as a natural sponge, a cellulose fiber sponge or a foamed plastic polymer sponge. For example, pad 140 may be made from a sponge-like material such as foamed polyurethane, and may be made of a thickness/depth in the range of about 15 mm to about 50 mm. Pad 140 may have a thickness/depth that is the same or about the same depth as first chamber 120. Pad 140 may also be formed of other suitable materials including natural or synthetic rubbers. Pad 140 may comprise an array of openings 142 some or all of which may be vertically aligned with openings 128 in plate 122. Fluid substance 119 may thus be forced through mesh openings in mesh screen 121 and through aligned openings 128 of plate 122 and pad 140. Depending upon the material from which pad 140 is made, and the fluid substance 119 that is being dispensed, the fluid substance may pass into and disperse within pad 140 for application to a skin surface, without the requirement of pad 140 having any specifically formed and/or defined openings into pad 140 that are aligned with openings 128 in plate 122. Pad 140 may be inherently sufficiently porous to permit the passage of fluid substance 119 into pad 140. In other embodiments, openings 142 may be formed and clearly defined within pad 140 and fluid substance 119 may flow into such openings when the fluid substance is forcibly discharged from first chamber 120. Each of the openings 142 in pad 140 may be aligned with openings 128 in plate 122. Openings 142 may also be conically tapered so as to narrow in an outward flow direction to modulate the flow characteristics of fluid substance 119 through pad 140 (FIG. 2C) and onto the skin surface of a person. Fluid substance 119 may partially seep and be at least partially absorbed into the material surrounding openings 142, which would then assist the operator when applying fluid substance 119 to a person's skin. Fluid substance 119 absorbed into the material surrounding openings 142 may pass to the outward surface of pad 140 and thus be transferred to a person's skin surface as applicator 110 is moved relative to a person's skin surface. The entire outward facing surface area of pad 140 may be utilized to apply fluid substance 119 to broad/large skin surface areas.

Pad 140 may be fixedly attached against the outward facing interface surface of plate 122 such as with a suitable adhesive or another attachment mechanism. In some embodiments, pad 140 is removable for washing and/or replacement and pad 140 can be re-attached to plate 140 with a suitable releasable attachment mechanism such as a hook-and-loop fastener (e.g. Velcro™) mechanism to plate 122 to facilitate reuse/replacement.

Still with particular reference to FIGS. 2A-2C, applicator 110 may also comprise a second chamber 150 which may be positioned vertically adjacent to first chamber 120 and on the opposite side of first chamber 120 to pad 140. Second chamber 150 may define a cavity in which may be positioned some of the components of a fluid pressure increasing mechanism or a force application mechanism as described hereinafter.

The components of second chamber 150 may be made from one or more suitable materials such as for example aluminum or a suitable plastic (e.g. polypropylene or high density polyethylene).

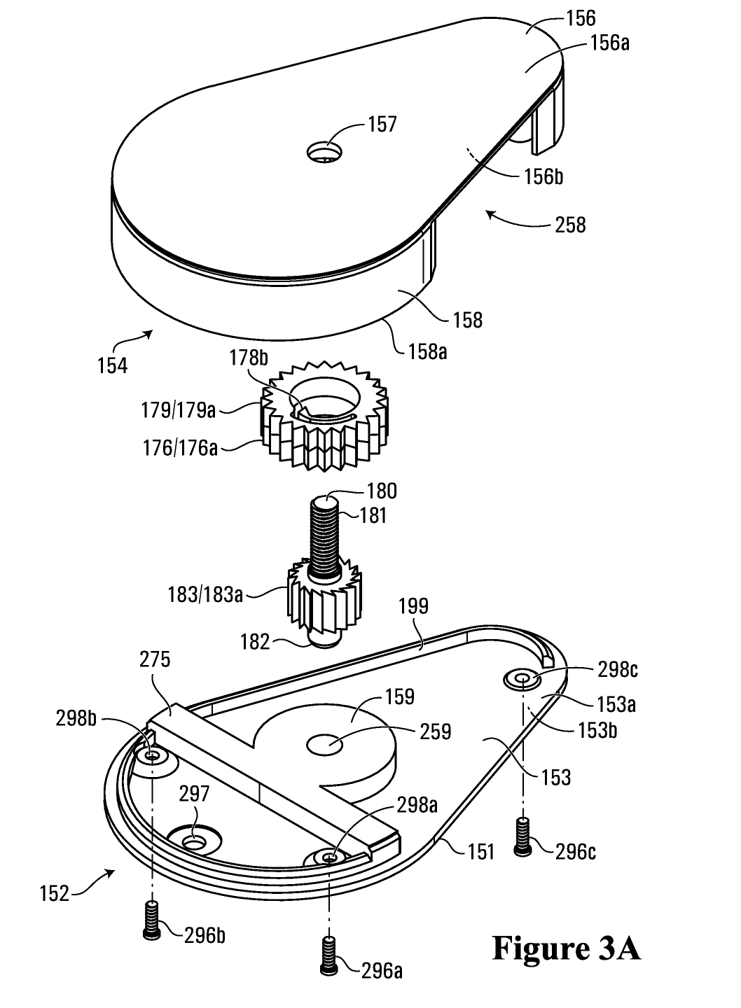

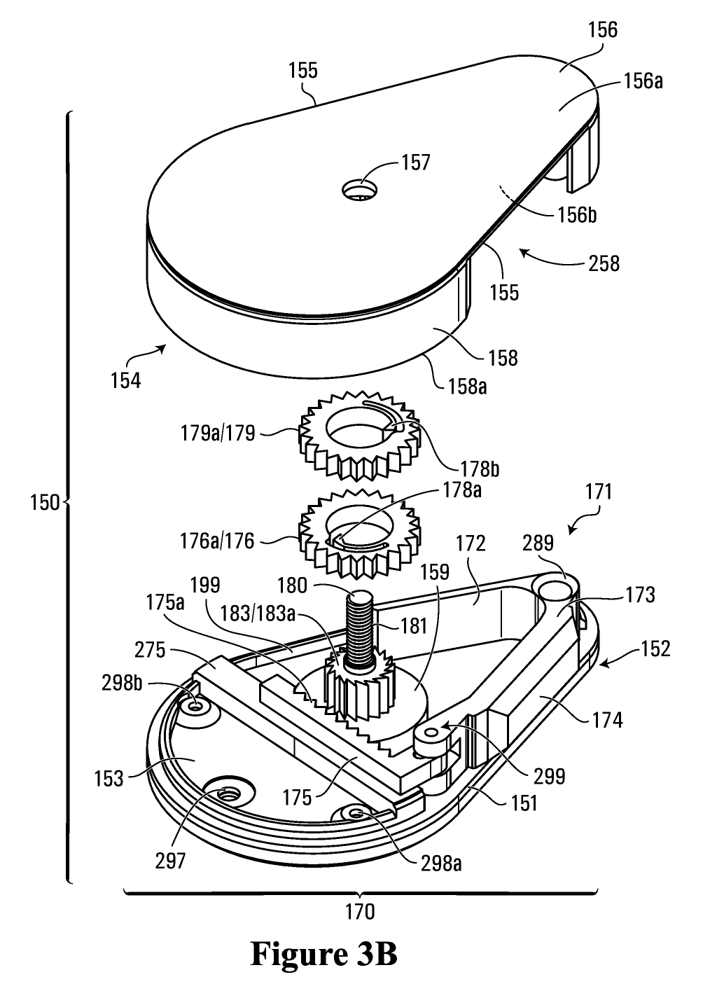

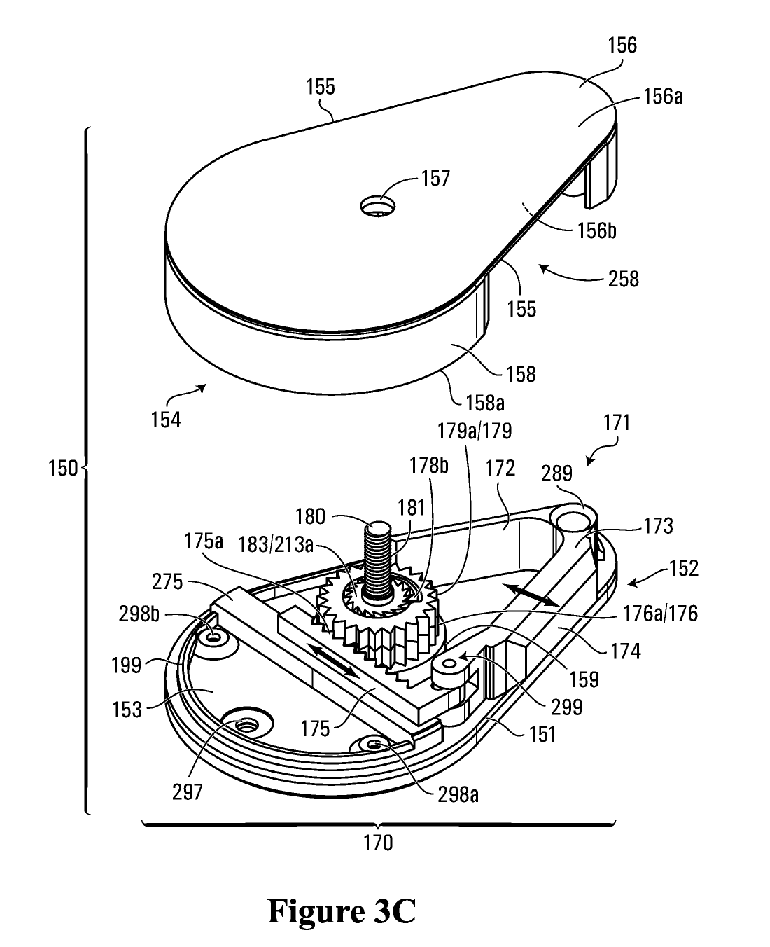

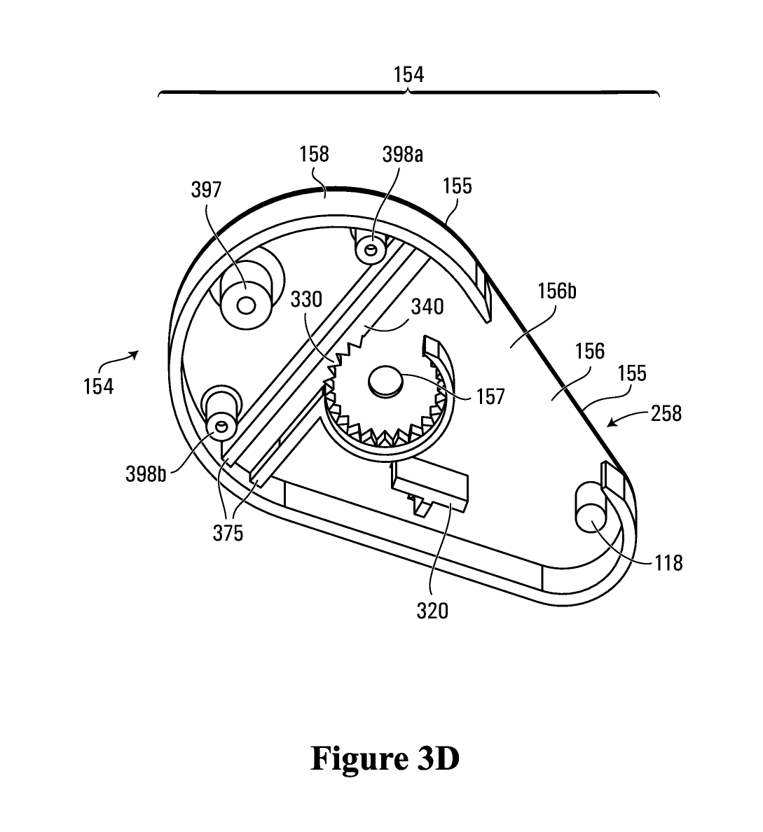

With reference to FIG. 2A, and FIGS. 3A-D, second chamber 150 may comprise a base 152 and a cover 154. Base 152 may be secured to cover 154, at least in part, by an interference friction fit or a snap fit between a lower outer edge of cover 154 and a raised edge 199 of base 152. Base 152 may alternately/also be secured to cover 154 by fasteners 296a-c (FIG. 3A), which may be screws. Base 152 may comprise a base plate 153 having a first surface 153a and a second surface 153b with openings 298a-c (which may be threaded) passing there between. Likewise, cover 154 may comprise a cover plate 156 with a first inward facing surface 156a and a second opposite outward facing surface 156b with threaded openings 398a-398c passing there between (FIG. 3D). Openings 298a-c and corresponding aligned openings 398a-c are for receiving there through fasteners 296a-c when base 152 is attached to cover 154 to secure base 152 to cover 154.

Cover plate 156 also has a sidewall 158 extending perpendicularly therefrom and has a lower edge 158a. Both cover plate 156 and base plate 153 may be sized, configured and oriented in a similar and co-operative fashion to plate 122.

Base 152 may be alternately/additionally attached to cover 154 with other securement mechanisms such as for example a suitable adhesive may be applied between the interfacing surfaces of lower edge portion of sidewall 158 and edge 151 of base plate 153 to provide/enhance the strength of, the connection. Alternately/additionally, depending upon the selection and suitability of the materials from which the parts are made, the interface between lower edge 158a of sidewall 158 and edge 151 of base plate 153 may be welded, brazened or soldered together.

Cover plate 156 may comprise a centrally-located internal circular edge 157 which defines an annular opening for receiving there through a rotatable member such as a threaded member 180. Cover plate 156 may further comprise an external perimeter edge 155 that provides a lip/flange that extends transversely outward (FIGS. 2B and 2C). Sidewall 124 of first chamber 120 may include a lower perimeter edge portion 127 (FIG. 2A) that may include an inward facing recess. Sidewall 124 may be elastically deformable to allow the recess of edge portion 127 to receive the lip/flange of top edge 155 of cover plate 156 and thus first chamber 120 may snap into place and be held in connection with second chamber 150. In addition, as part of the interfacing surface forces holding lower edge portion 127 in engagement with lip/flange 155, friction forces acting between the interfacing surfaces of the recess of lower edge portion 127 of sidewall 124 and lip/flange of top edge 155 of cover plate 156 may also assist in holding the lip/flange of top edge 155 in the recess of lower edge portion 127 of sidewall 124. In this way, first chamber 120 may be secured to second chamber 150 and may resist forces that may tend to push first chamber 120 and second chamber 150 apart during operation of applicator 110. One or more alternate or additional securement mechanisms for securing the connection between first chamber 120 and second chamber 150 are also contemplated. For example, a suitable adhesive may be applied between the interfacing surfaces of the recess of lower edge portion 127 of sidewall 124 and lip/flange of top edge 155 of cover plate 156 to enhance the strength of the connection. Alternately/additionally, depending upon the suitability of the materials, the interface between lower edge portion 127 of sidewall 124 and the top edge 155 of cover plate 156 may be welded, brazened or soldered together.

Applicator 110 may also comprise a press plate assembly 130 (FIG. 2A) that may be part of a pressure increasing mechanism for increasing the pressure within the cavity of first chamber 120 and to force fluid substance 119 towards the openings 128 in plate 122. Press plate assembly 130 may be located inside and movable within the interior cavity provided by first chamber 120, towards and optionally also away from the openings 128 in plate 122. When applicator 110 is completely assembled, first chamber 120 contains fluid substance 119, and press plate assembly 130 can be initially positioned adjacent first surface 156a of cover plate 156 such that fluid substance 119 is held within the interior cavity of first chamber 120 sandwiched between: (a) press plate assembly 130 and (b) the combination of a mesh screen 121/plate 122/pad 140, and contained at the sides within the interior cavity by continuous sidewall 124 of first chamber 120.

Press plate assembly 130 may for example be a single plate or for example may be comprised of a first press plate 132 and a second press plate 134. First press plate 132 and second press plate 134 may be sized, dimensioned and oriented in a similar fashion to plate 122, such that press plate assembly 130 can translate through the interior cavity of first chamber 120 towards plate 122 while maintaining a sealing contact with continuous sidewall 124 and threaded member 180. A centrally located threaded opening 133 may extend through first press plate 132 and may be surrounded on one face of first press plate 132 by a raised annular lip 135. The outer perimeter of first press plate 132 may be defined by an external edge 131.

The outer perimeter of second press plate 134 may be defined by an external edge 137, and may have a centrally-located internal circular edge 136 which defines an opening for receiving raised annular lip 135 of first press plate 132. Thus press plate assembly 130 can be assembled by pressing first press plate 132 and second press plate 134 together while aligning external edges 131 and 137, such that internal edge 136 frictionally engages with an outward facing surface of the raised annular lip 135 providing an interference fit to hold first press plate 132 and second press plate 134 together. Other securement mechanisms may be provided to hold first press plate 132 to second press plate 134.

First press plate 132 and second press plate 134 may each, independently, be made from one or more suitable materials such as aluminum, rubber or a suitable plastic (e.g. polypropylene or high density polyethylene). For example, first press plate 132 may be made from a relatively rigid material (e.g. a hard plastic) to provide a strong base member. Second press plate 134 may be made from a more flexible and ductile material (e.g. rubber) that may be suitable to provide a sealing member that can maintain a seal with the inner surface of continuous sidewall 124 of first chamber 120, including when press plate assembly 130 is exerting additional pressure on fluid substance 119 to force the fluid substance through screen 121 and plate 122 into pad 140.

External edges 137 and 131 may be concaved or convexed to facilitate sealing engagement with the inner surface of continuous sidewall 124. One or both of external edges 137 and 131 may also be configured to retain one or more O-ring-type gaskets to provide sealing engagement with continuous sidewall 124 including during movement of press plate assembly 130 through the cavity in first chamber 120.

With particular reference to FIGS. 2A-2C, it can be appreciated that spiral threaded opening 133 receives compatibly spiral threaded member 180 such that rotation of threaded member 180 in one rotational direction (e.g. clockwise) causes the relative translational movement of press plate assembly 130 through the cavity of first chamber 120 towards plate 122. This places fluid substance 119 under an increase in positive pressure thereby forcing at least a portion out of first chamber 120 through screen 121 and plate 122 and into pad 140. Once in pad 140, fluid substance may be located for application by an operator for application to a skin surface.

In FIGS. 2B and 2C, typical translational movement of press plate assembly 130 and fluid substance 119 are shown with bold-type arrows. As the rotation of threaded member 180 can be effected by the user/operator, this provides a mechanism for forcing and moving fluid substance 119 out through screen 121, and arrays of openings 128 and 142 for applying to a person's (such as the operator's) skin surface.

Referring to FIGS. 3A-D, in some embodiments of applicator 110, rotation of threaded member 180 may be effected by a rotational drive mechanism such as for example an operator-actuated ratchet movement mechanism 170 which may be housed in second chamber 150 and may include a ratchet member. In some embodiments, operator-actuated ratchet movement mechanism 170 may comprise an elastically deformable device such as a V-bracket 171 with a first lever arm 172 and a second lever arm 173 (FIGS. 3B and 3C). At the location where first lever arm 172 connects with second lever arm 173, V-bracket 171 defines a cylindrical opening 289 which is sized, configured and oriented to receive a co-operating cylindrical peg/pin member 118 (FIG. 3D). Peg/pin member 118 may be fixedly attached to/integrally formed with, and extend perpendicularly inwards from, second surface 156b of cover plate 156. The combination of peg/pin 118, opening 289, and first surface 153a of base plate 153 limits the movement of V-bracket 171 to pivotal movement.

Second lever arm 173 may form a pressable/releasable button 174 which may be positioned along edge 151 of base plate 153. Sidewall 158 may be partitioned with an opening 258 to accommodate second lever arm 173 and button 174 such that an overall substantially continuous outward facing side surface is provided.

In some embodiments, first lever arm 172 may abut a backing projection 320 which may extend inwardly from, and perpendicular to, second surface 156b of cover plate 156 (FIG. 3D). Backing projection 320 may restrict movement of first lever arm 172 when button 174 is pressed. V-bracket 171 may be formed of a flexible material, such as a suitable hard elastically deformable plastic, which flexes when button 174 is pressed. The material of V-bracket 171 may be elastically deformable, such that it returns to its original position once the user releases pressure from button 174. Thus, button 174 may be repeatedly pressable.

The distal end of second lever arm 173 may be fashioned in a clevis and pin style connection 299 (FIGS. 3B and 3C) to pivotally connect second lever arm 173 to a sliding bar 175, such that pressing button 174 results in translational inward movement of sliding bar 175, and releasing button 174 causes sliding bar 175 to translate back in an outward direction to its original position. In an embodiment, first surface 153a of base plate 153 may comprise a bar-shaped platform 275 along which sliding bar 175 can translate. In an embodiment, second surface 156b of cover plate 156 may comprise runners/channel walls 375 (FIG. 3D), which are positioned to receive sliding bar 175 by forming a channel with platform 275. This arrangement restricts the movement of sliding bar 175 to translational inward and outward movement.

Sliding bar 175 may be made of one or more suitable materials such as for example a lightweight metal material or a hard plastic, and may comprise a plurality of symmetric teeth 175a which are for engaging a plurality of symmetric gear teeth 176a on a first outer gear member 176 (FIG. 3B).

The gear drive mechanism may be operable to drive a gear mechanism. The gear mechanism may comprise a first outer gear member 176 that may have at least one inwardly projecting pawl 178a for engaging a plurality of asymmetric ratchet teeth 183a on an asymmetric inner gear member 183. With this arrangement, pressing button 174 inwardly translates sliding bar 175 inwards, which rotates first outer gear member 176 in a clockwise direction about a vertical axis through threaded member 180 (in the orientation of FIG. 3C). This motion causes pawl 178a to abut and engage the angled radial face of a tooth of plurality of asymmetric ratchet teeth 183a. This imparts a generally tangential force on asymmetric gear member 176, causing gear member 183 to also rotate in a clockwise direction. This rotation of gear member 183 causes threaded member 180 to rotate in a clockwise direction and by virtue of the orientation of the threads in threaded member 180 and threaded opening 133 of press plate 132, press plate assembly 130 will move in the translational direction in the cavity of chamber 120 shown in FIGS. 2B and 2C.

By releasing button 174, V-bracket 171 will cause second lever arm 173 to exert a force that causes the outward translation of sliding bar 175, which rotates first outer gear member 176 in a counter-clockwise rotational direction about the vertical axis through threaded member 180 (in the orientation of FIG. 3C). However, this motion causes pawl 178a to slide over the radial faces of the teeth of plurality of asymmetric ratchet teeth 183a. Thus, while a generally tangential force is imparted on asymmetric gear member 176, this does not cause gear member 183 to also rotate in a counter-clockwise direction and thus threaded member 180 will not be rotated. Accordingly, there will be no reverse/backward movement of press plate assembly 130 within the cavity of first chamber 120.

In some embodiments, asymmetric gear member 183 may be integral to, or fixedly attached to, threaded member 180. Threaded member 180 may for example be 5-15 mm in diameter and may be formed of a strong and resilient material such as for example aluminum or a suitable plastic. Threaded member 180 may comprise a spiral threaded section 181 and a smooth section 182 (FIG. 3A). Asymmetric gear member 183, which may likewise be made from for example aluminum or a suitable plastic, may be oriented perpendicular and co-axial to the axis of threaded member 180 and may rotate with threaded member 180 about its vertical axis. Asymmetric gear member 183 may be fixedly attached to an exposed portion of smooth section 182 such that smooth section 182 extends vertically down beyond asymmetric gear member 183.

In some embodiments, base plate 153 may comprise, or have mounted thereto, a centrally located and raised platform 159 which in part defines a cylindrical slot 259 (FIG. 3A). Cylindrical slot 259 may be sized, configured and oriented to receive smooth cylindrical portion 182 of threaded member 180. This configuration may serve to restrict the relative axial movement of threaded member 180 while the interfacing cylindrical surfaces of threaded member 180 and slot 259 slide relative to each other allowing threaded member 180 to rotate freely around its vertical axis.

In some embodiments, the longitudinal height of asymmetric gear member 183 may be approximately twice that of first outer gear member 176, such that it can further accommodate a second outer gear member 179. Second outer gear member 179 may have an inner pawl 178b for also interfacing with asymmetric teeth 183a of asymmetric gear member 183. Second outer gear member 179 may also have outer teeth 179a for interfacing with a plurality of opposing teeth 330 formed in a surface of a retaining platform 340 of cover plate 156 (FIG. 3D). Retaining platform 340 may extend downwards from, and perpendicular to, second surface 156b of cover plate 156.

With this arrangement, pressing button 174 inwardly translates sliding bar 175 inwards, which rotates first outer gear member 176 in a clockwise direction (in the orientation of FIG. 3C). As indicated above, this motion causes pawl 178a to abut and engage the radial face of a tooth of plurality of asymmetric ratchet teeth 183a. This imparts a generally tangential force on asymmetric gear member 183, causing it to also rotate in a clockwise direction. This rotation of asymmetric gear member 183 causes threaded member 180 to rotate in a clockwise direction and by virtue of the orientation of the threads in threaded member 180 and opening 133 of press plate 132, press plate assembly 130 will move in the direction shown in FIGS. 2B and 2C. During clockwise motion of asymmetric gear member 183, asymmetric teeth 183a will slide past pawl 178b of gear member 179, which will not rotate relative about the axis of threaded member 180 due to the engagement of teeth 179a with teeth 330 formed in the surface of a retaining platform 340 of cover plate 156.

By releasing button 174, V-bracket 171 will cause second lever arm 173 to exert a force that causes the outward translation of sliding bar 175, which rotates first outer gear member 176 in a counter-clockwise direction (in the orientation of FIG. 3C). However, as indicated above, this motion causes pawl 178a to slide over the radial faces of the teeth of plurality of asymmetric ratchet teeth 183a. This does not cause asymmetric gear member 183 to rotate in a counter-clockwise direction and thus threaded member 180 will not be rotated. Accordingly, there will be no reverse/backward movement of press plate assembly 130. Additionally, during counter clockwise motion of first gear member 176, a tooth of asymmetric teeth 183a of asymmetric gear member 183 engages with pawl 178b of second gear member 179 which is fixed from rotating by the interaction of teeth 179a with teeth 330 of platform 340.

Retaining platform 340 may thus restrict/the rotation of second outer gear member 179 due to the opposition of teeth 330/179a. The interaction between pawl 178b and asymmetric teeth 183a may ensure that asymmetric gear member 183 can only rotate uni-directionally. As such, threaded member 180 may be limited to rotating uni-directionally and press plate assembly 130 may only move in one direction within the cavity of first chamber 120.

Taken together, the components of user-activated ratchet mechanism 170 allow for the uni-directional rotation of threaded member 180 by repeated pressing of button 174, and thus provide a means for advancing press plate assembly 130 through first chamber 120 in only one direction—towards plate 122.

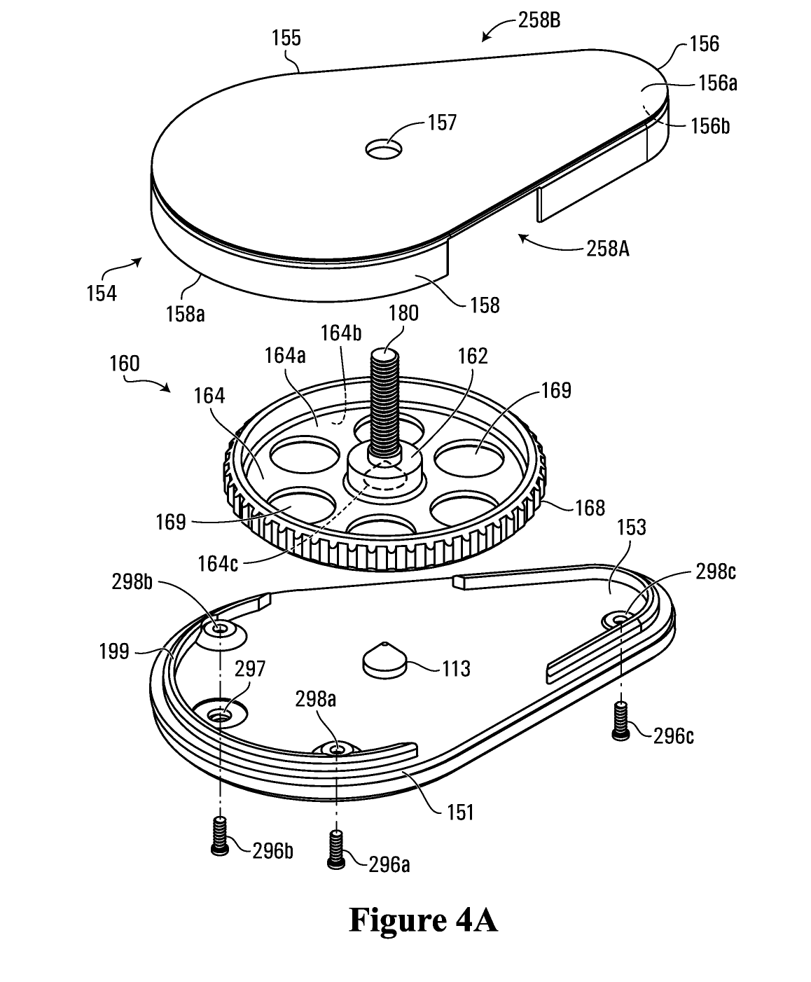

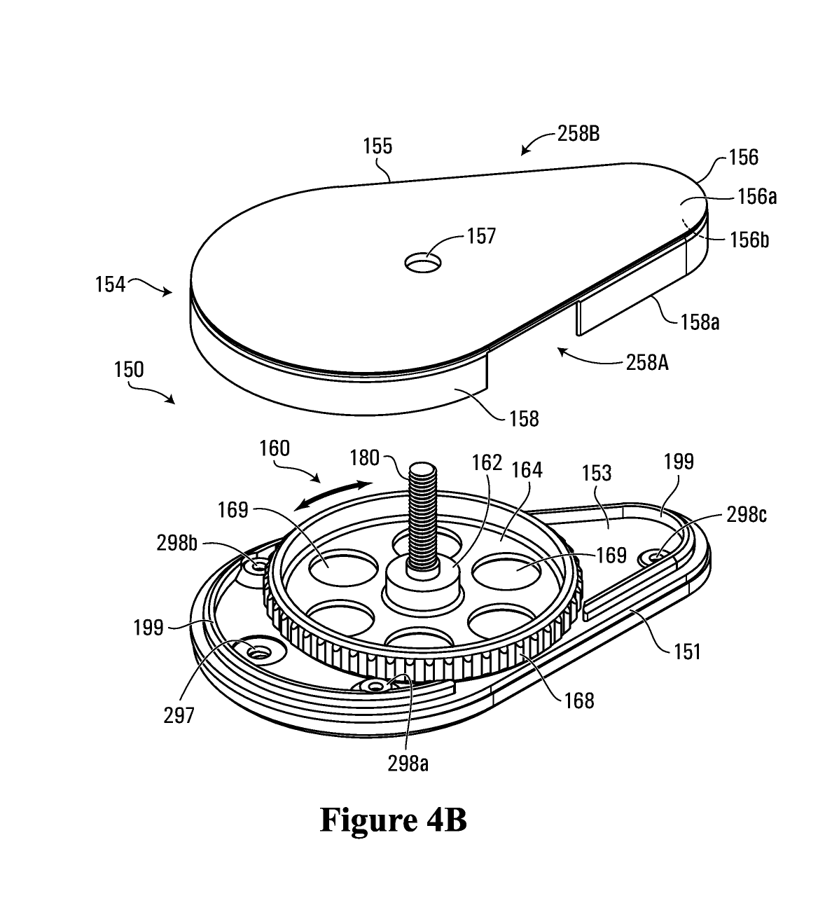

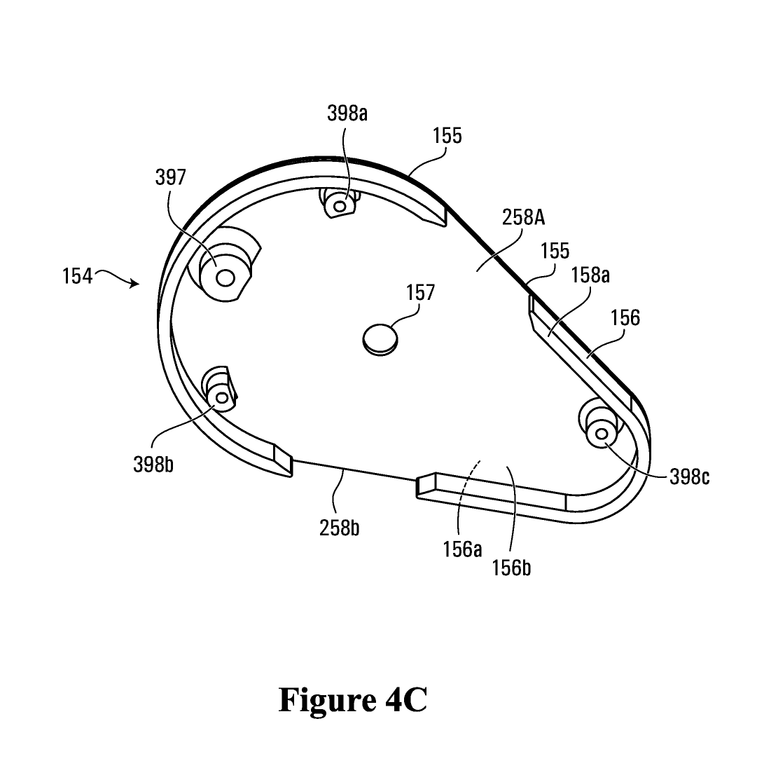

Referring now to FIGS. 4A-C, there is shown a configuration for second chamber 150 which is substantially the same as the embodiment shown in FIGS. 3A-3B, except as described hereinafter. Second chamber 150 comprises an alternate movement mechanism for effecting rotation of threaded member 180 of applicator 110 and thus the translational movement of press plate assembly 130 within first chamber 120. This alternate movement mechanism includes a rotatable turning wheel 160 for rotating threaded member 180 and thus for advancing press plate assembly 130 through first chamber 120. Turning wheel 160 may be sized, configured and positioned to rest on or just above base plate 153 with opposite side portions of the perimeter of turning wheel 160 extending beyond edge 151 of base plate 153. As best seen in FIG. 4C, opposite openings 258A and 258B in sidewall 158 provide access for an operator to turn wheel 160 when cover 154 is mounted in place on base plate 153 of second chamber 150. Turning wheel 160 may be made from any one or more suitable materials such as a hard plastic or a lightweight metal, and may comprise a generally circular plate 164 having inner/upward and outer/downward facing surfaces 164a and 164b which may be oriented substantially parallel to base plate 153. Circular plate 164 may have angularly spaced vertical openings 169 there through. Centrally located on inner facing surface 164a may be a raised mounting platform 162 to which threaded member 180 may be fixedly mounted and connected. The fixed connection between mounting platform 162 and threaded member 180 will be adapted so that torque can be transferred between turning wheel 160 and threaded member 180. The connection may for example be made by application of a suitable adhesive between adjacent surfaces. Additionally/alternatively, the connection may be made by providing a suitably shaped opening in mounting platform 162 engaged by a correspondingly shaped head on threaded member 180 (e.g. they could both be hexagonally shaped) so that that torque can be transferred from mounting platform 162 to the head of threaded member 180.

Centrally located on outer facing surface 164b is a recess 164c within platform 162 for receiving a centrally located generally conically shaped projection 113 of base plate 153 (FIG. 4A). Projection 113 provides a conically shaped bearing surface that slidingly/rotatingly engages with a compatible inner surface of recess 164c of turning wheel 160 and allows turning wheel 160 (and thus threaded member 180 that is secured thereto) to rotate, while restricting axial movement, in relation to base plate 153.

The perimeter of circular plate 164 may be defined by an outward facing circumferential sidewall 168 extending perpendicularly to base plate 153. Side wall 168 may have an outer surface that may be textured to facilitate rotation by the user. In some embodiments, sidewall 158 is partitioned to provide opposed openings 258A and 258B which can accommodate sidewall 168 of turning wheel 160 to extend there through such that the user can rotate turning wheel 160 by running his/her fingers along sidewalls 158/168. Because rotation of turning wheel 160 in for example a clockwise direction about the axis of threaded member 180 results in rotation of threaded member 180, rotation of turning wheel 160 is operable to advance press plate assembly 130 through first chamber 120, thereby discharging fluid substance 119 from first chamber 120.

In some embodiments turning wheel 160 can be rotated in both clockwise and counter-clockwise rotational directions about the axis of threaded member 180. Thus, by rotating turning wheel 160 in one rotational direction, press plate assembly 130 can be moved to exert additional pressure on fluid substance 119 to expel the fluid substance 119 through screen 121 and plate 122 into pad 140. By rotating turning wheel 160 in an opposite rotational direction, press plate assembly may be moved backward within the interior cavity of first chamber 120 to thus remove additional pressure applied to fluid substance 119. Additionally, in some embodiments, this may permit first chamber 120 to be re-filled with fluid substance 119, if a port into the cavity is provided which can be opened to allow refilling and then closed to secure the fluid substance within the cavity of first chamber 120.

In some embodiments, the rotational direction of turning wheel 160 may be restricted to only one direction that advances press plate assembly 130 within first chamber 120 towards plate 122 to exert additional pressure on fluid substance 119 to expel it through screen 121 and plate 122 into pad 140. Rotational movement in the opposite direction may be restricted for example by a pawl device (not shown) mounted on base 153 and which may engage gear teeth on a suitably formed outer surface 168 of turning wheel 160.

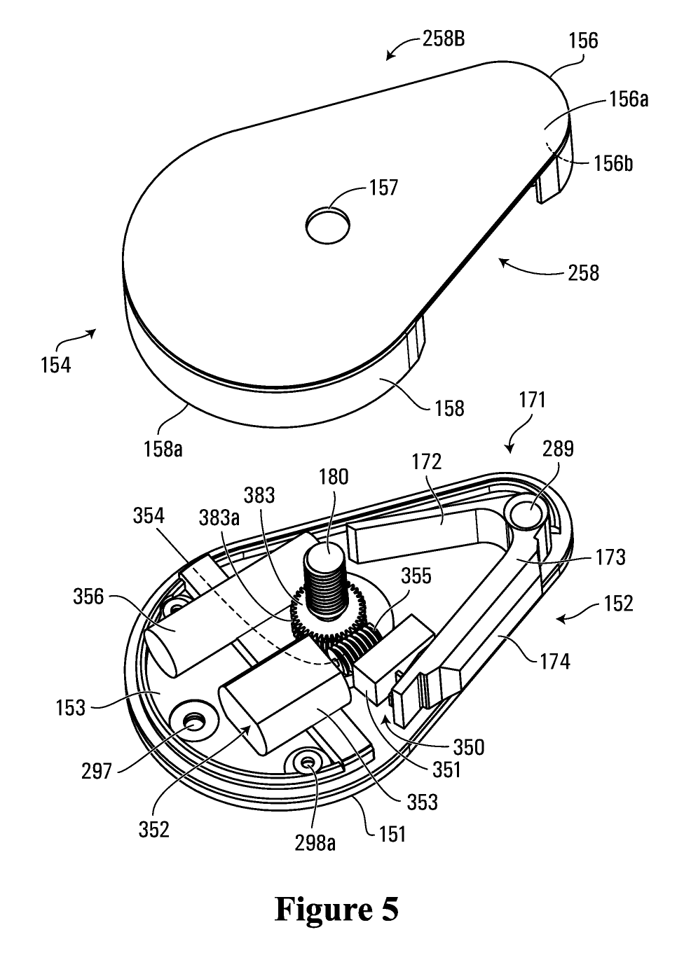

Referring now to FIG. 5, second chamber 150 may comprise an electric drive mechanism 350 for rotating threaded member 180 to advance press plate assembly 130 through the cavity of first chamber 120. Electric drive mechanism 350 may comprise a switch 351 which is configured and oriented to be activated by movement of second lever arm 173 when the user presses button 174. Switch 351 may be, for example, a contact type switch.

Electric drive mechanism 350 may further comprise a motor assembly 352, which may comprise a motor 353 configured to drive a rotatable shaft such as a motor shaft 354 having a spirally threaded extension 355. Motor 353 may be, for example a brush or brushless DC motor having a voltage in the range of 0.5 to 3.0 volts.

The threads of spirally threaded extension 355 may be sized and configured to engage with a plurality of teeth 383a of a gear member 383 which is fixedly connected, or integral, to threaded member 180. In this arrangement, actuation of motor 353 drives the rotation of threaded extension 355 about the longitudinal axis of motor shaft 354, which causes the rotation of threaded member 180.

In an embodiment, electric drive mechanism 350 further comprises a source of power such as a battery 356, which may be electrically connected to motor assembly 352 and switch 351. Battery 356 may be, for example, a button cell type battery or an AAA alkaline battery.

Switch 351 may be physically activated and de-activated by an operator who pushes on and releases pressable button 174 as described above to close and then open the switch and thus the electrical circuit causing motor 353 be turned on and off. If desirable, an electronic speed controller might be added to the circuit so that the speed of the motor can be suitably set and optionally varied by an operator.

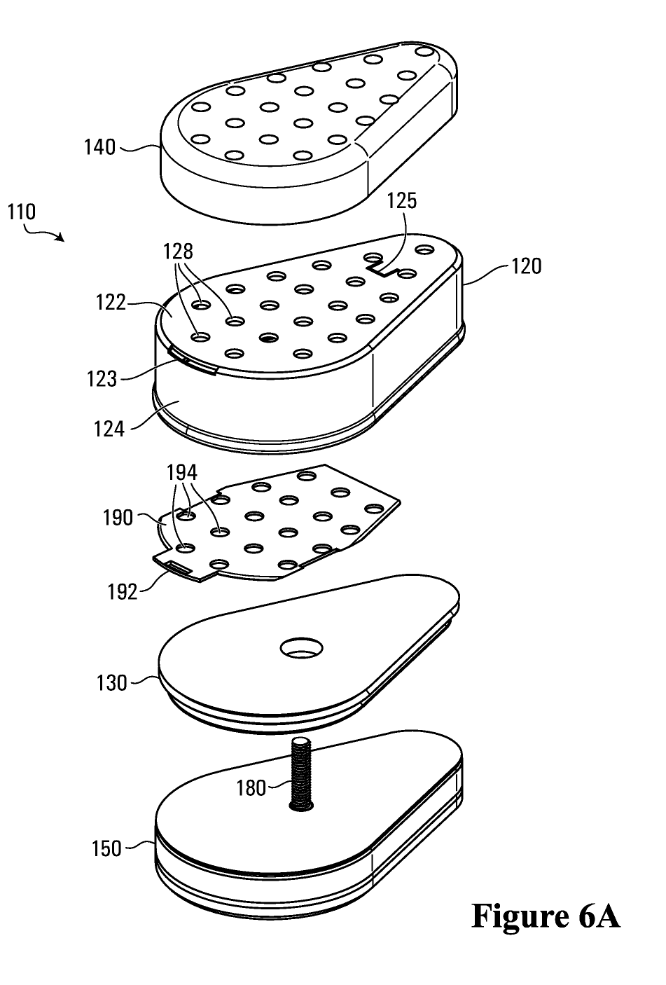

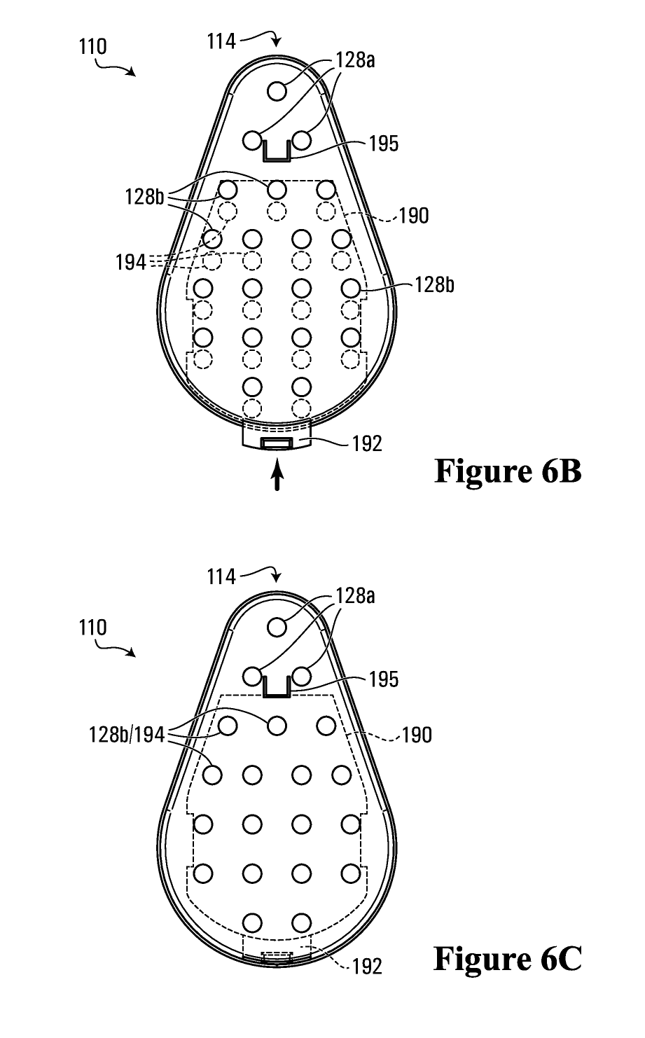

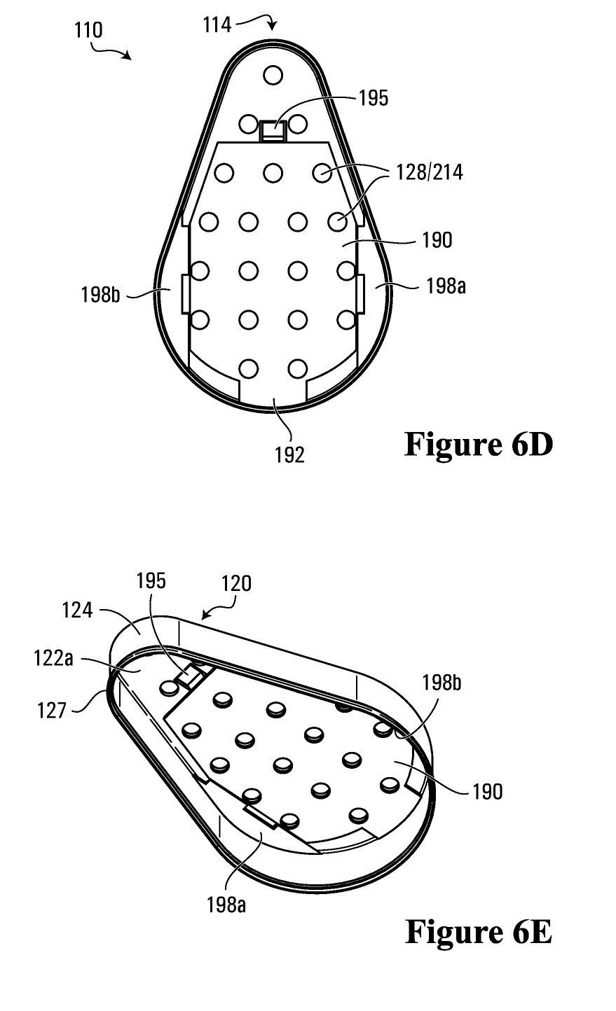

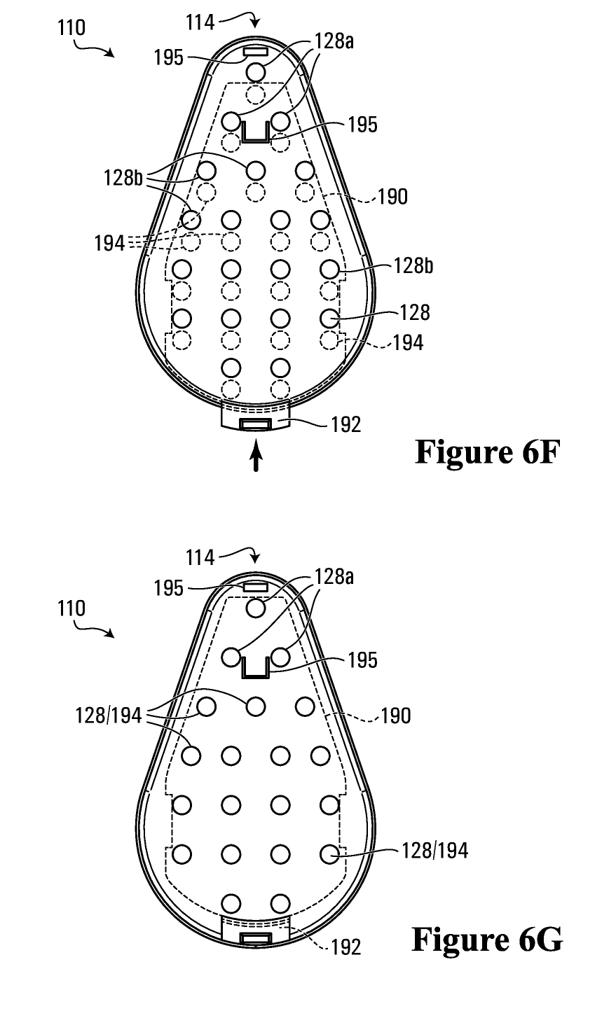

Referring now to FIGS. 6A-6E, applicator 110 may also comprise a movable plate device comprising a flow restrictor plate 190. Flow restrictor plate 190 may be positioned inside first chamber 120 and may be located adjacent to, and interface directly with an inward facing surface 122a (FIG. 6E) of plate 122. In some embodiments, a screen like mesh screen 121 (FIGS. 2A and 2B) may also be provided between flow restrictor plate 190 and the inward facing surface 122a of plate 122.

Flow restrictor plate 190 may be generally sized and configured in a manner that is similar to plate 122, but may be configured to allow for translational longitudinal movement within first chamber 120. For example, as shown in FIGS. 6A to 6E, flow restrictor plate 190 may be truncated at one longitudinal end such that it does not span the entire length of plate 122. Opposite the truncated end, flow restrictor plate 190 may include a tab member 192 which may extend through an opening 123 in sidewall 124. A tight fit and/or other sealing mechanism should be provided between the surfaces defining opening 123 and the surface of tab member 192 to prevent fluid substance 119 from leaking out through opening 123. A sealing device (e.g. a rubber sealing ring) may be installed and held in opening 123 and may engage the surfaces of flow restrictor plate 190 to provide such a seal.

Flow restrictor plate 190 can move with linear, reciprocating sliding motion in a longitudinal direction and can be moved between a closed position (FIG. 6B) and an open position (FIG. 6C) by an operator pushing/pulling tab 192. Flow restrictor plate 190 may be made of one of more suitable materials such as aluminum or a suitable plastic (e.g. polypropylene or high density polyethylene).

Plate 122 may have an inward extending protrusion 125 which limits the extent of inward linear, longitudinal movement of flow restrictor plate 190 when tab 192 is pushed in.

Flow restrictor plate 190 may have an array of openings 194 which are configured and arranged, and may be positioned to be at least partially, and preferably completely, vertically aligned with openings 128b of the array of openings 128 on plate 122 when flow restrictor plate 190 is in the open position (FIG. 6C). In the open position, flow restrictor plate 190 has an end portion that abuts an abutment 195 to ensure proper longitudinal positioning of flow restrictor plate 190 relative to plate 122. In this open position, a first group of openings in plate comprising all openings 128a, 128b on plate 122, including openings 128b that interface with restrictor plate 192 are open to flow of fluid substance 119 (i.e. a group comprising no openings 128a, 128b are blocked). Thus, in the open position, fluid substance 119 is not restricted from flowing through any of openings 128a or 128b, and thus fluid substance 119 will distribute evenly into pad 140 during use.

When flow restrictor plate 190 is moved to the closed position (FIG. 6B) by an operator engaging tab 192 (which protrudes sufficiently from first chamber 120 to allow engagement by an operator), the end portion of flow restrictor plate 190 is positioned away from an abutment 195. Tab 192 can only be pulled outwards a limited amount, determined by when the opposite end of flow restrictor plate 190 abuts the inner surface of chamber 120. Pulling tab 192 out as far as it will go will ensure proper longitudinal positioning of flow restrictor plate 190 relative to plate 122. In this closed position, openings 194 of flow restrictor plate 190 are positioned such that fluid substance 119 can no longer flow through the first group of openings 128b. Fluid substance 119 can only flow through a second group of openings comprising a selected number of openings 128a of the array of openings 128 in plate 122 as openings 128b are blocked.

In the closed position, openings 128b in plate 122 that interface with flow restrictor plate 194 are not aligned with openings 194 in flow restrictor plate 190 and thus flow of fluid substance 119 through such openings 128b in plate 122 is blocked. However, some openings 128a in plate 122 proximate the end tip region of applicator 110 do not interface with flow restrictor plate 194. So the movement of restrictor plate 194 has no effect on the flow of fluid substance 119 through openings 128a. In this position, fluid substance 119 is blocked from flowing through openings 128b of the array of openings 128 which are covered by the body portion of flow restrictor plate 190. In this case, fluid substance 119 is only able to flow through a front end tip region of plate 122.

Thus, where applicator 110 including plate 122 is wedge shaped, flow restrictor plate 190 does not block openings 128a which are generally at or proximate the end tip portion 114 of plate 122, in the open or closed positions. The user can selectively communicate fluid substance 119 into pad 140 either evenly throughout pad or only in the region of end tip portion 114 by positioning flow resistor plate 190 in the respective open or closed positions. This configuration may be useful for applying the fluid substance to smaller or constricted areas of a person's skin such as behind a person's ears or only on a person's nose.

With reference to FIGS. 6F and 6G, in this embodiment, in the open position (FIG. 6G), all of the openings 128 in plate 122 are aligned with openings 194 in flow restrictor plate 190 thus allowing fluid substance 119 to flow evenly throughout pad 140. In the closed position (FIG. 6F), none of the openings 128 in plate 122 are aligned with openings 194 in flow restrictor plate 190 thus blocking any fluid substance 119 from flowing into pad 140. Such an arrangement may be desirable for example where fluid substance 119 has a low viscosity. With a low viscosity fluid substance 119, even when the pressure increasing mechanism is not being activated by an operator (e.g. during storage) plate 122, optional mesh screen 121 and pad 140 may not be able to prevent seeping out of fluid substance 119 from pad 140 when such seepage is undesired. Thus, flow restrictor plate 190 may be configured to be utilized as a barrier against unwanted leakage of fluid substance 119.

With reference to FIGS. 6D and 6E, a pair of guide rails 198a, 198b may be mounted within the cavity of first chamber 120 spaced a small amount from the underneath surface 122a of plate 122. Guide rails 198a/198b may respectively engage opposite, lower side edge surface portions/edges of flow restrictor plate 190 and thus may hold flow restrictor plate 190 in a generally parallel relationship to lower planar surface 122a of plate 122. In this way, guide rails 198a, 198b may assist in guiding flowrestrictor plate 190 as it moves between open and closed positions.

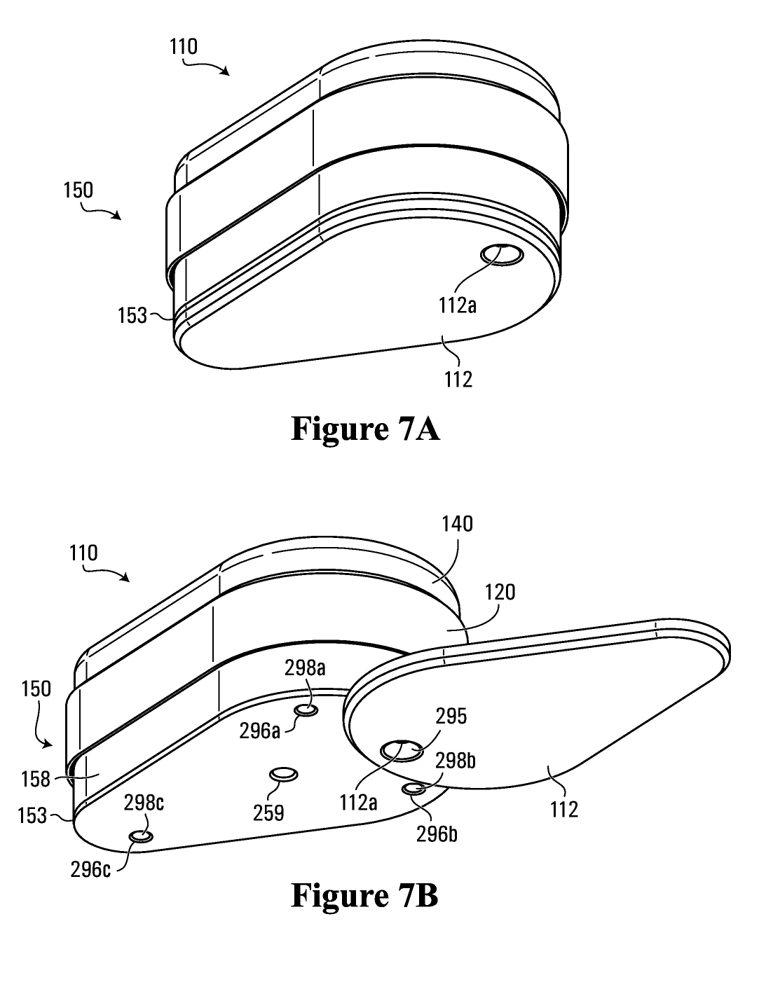

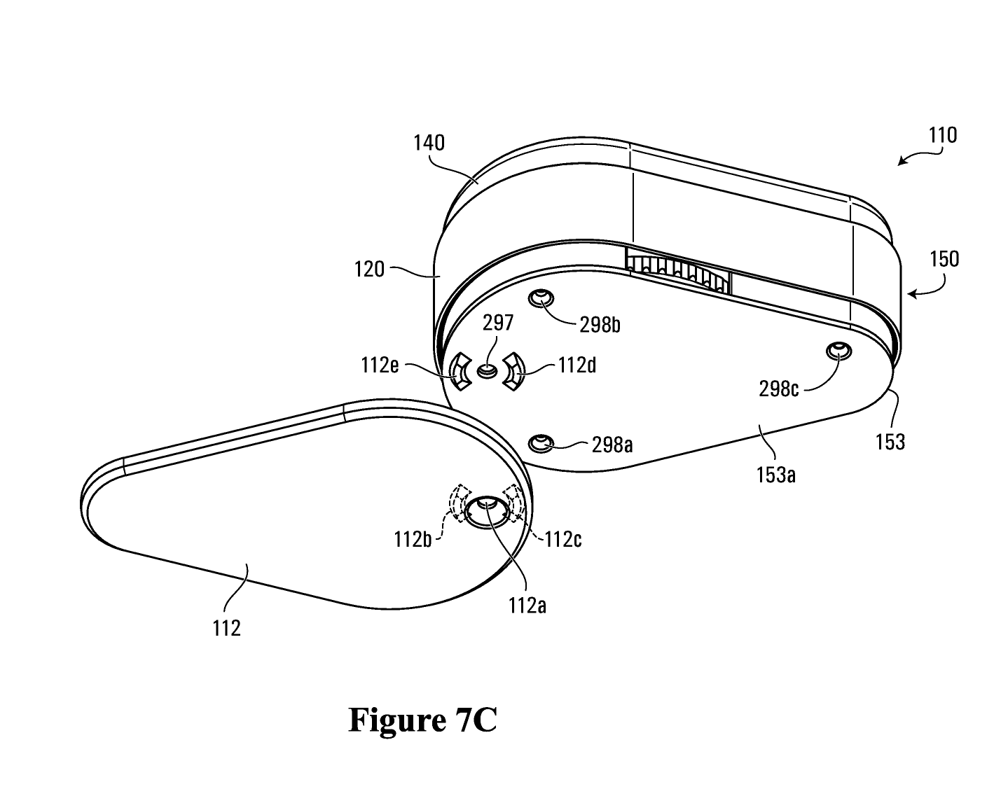

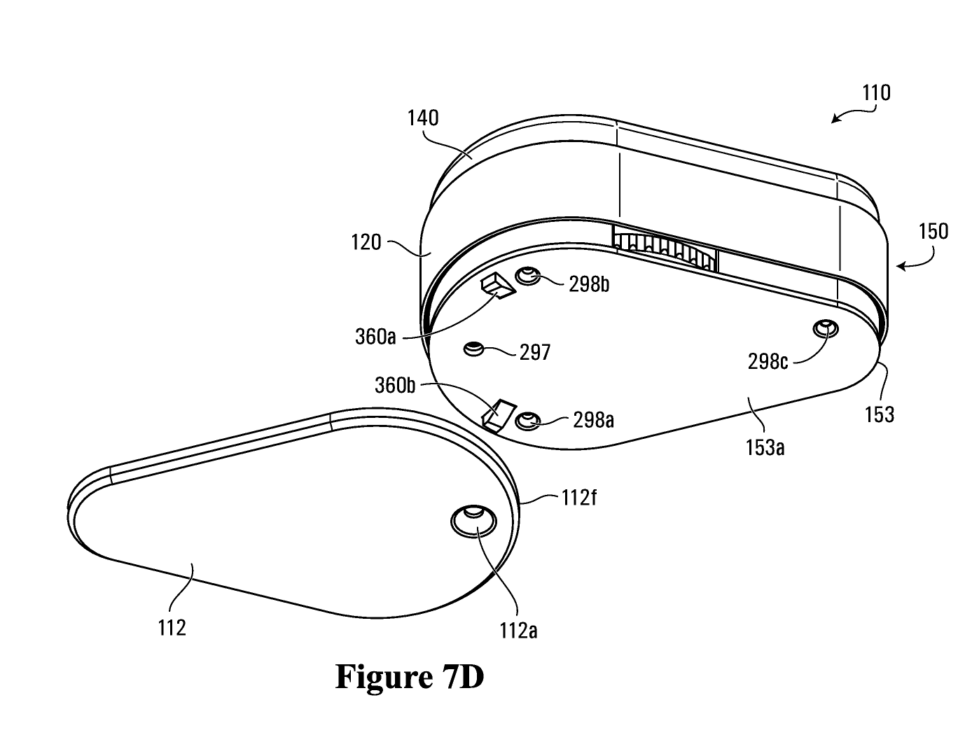

Referring to FIGS. 7A-7D, there is shown an embodiment of applicator 110 which comprises a handle 112 for handling during the application of fluid substance 119 to the user's skin. In FIG. 7A, handle 112 is in the closed position, and is shown as a plate which is sized, shaped and positioned to underlie base plate 153 (i.e. have an outer perimeter shape profile which corresponds generally with/mirrors the outer perimeter profile of base plate 153). In this position, handle 112 is conveniently positioned for packing or storage or just when generally not required for use of applicator 110. In FIG. 7B, handle 112 is shown in an operational, open position. This position is achieved by rotation of handle 112 approximately 180 degrees about a hinged fastener 112a which is inset in a recess 295 secured, through opening 297 in base plate 153, to a receiver 397 on surface 156b of cover 154 (FIGS. 3D and 4C). This connection serves to pivotally mount handle 112 to base plate 153. In the open position, handle 112 can extend a user's/operator's effective reach, thereby facilitating the application of fluid substance 119 to hard-to-reach areas of the user's/operator's own skin surface such as the middle of the operator's back.

As shown in FIG. 7C, in an embodiment, handle 112 can be releasably locked in both the open and closed positions by a releasable locking mechanism such as a set of complimentary projections/depressions. For example, the inner facing surface of handle 112 may comprise upstanding resilient projections 112b and 112c, which may be substantially identical and which may be sized and oriented to be receivable in proximal and distal depressions 112d and 112e. Depressions 112d/e may be positioned on opposite sides of opening 297 along the bilateral axis of base plate 153. Depressions 112d/e may be sized slightly larger than projections 112b/c in order to easily accommodate them therein. The occupation of proximal depression 112d by projection 112c (and depression 112e by projection 112b) may releasably lock/hold handle 112 in the open position, while the occupation of proximal depression 112d by projection 112b (and depression 112e by projection 112c) may releasably lock/hold handle 112 in the closed position. Rotation of handle 112 about fastener 112a may allow for transitions between the open and closed positions owing to the slightly flexible nature of the materials of handle 112 and the allowance of the connection made by fastener 112a.

Referring now to FIG. 7D, in an embodiment, handle 112 may be reversibly-fixed in the open position by depressable wedges 360a and 360b. In the closed position, handle 112 overlays wedges 360a/b which are depressed to be substantially flush with surface 153a of base plate 153. In the open position, wedges 360a/b project above surface 153a, and they abut an edge 112f of handle 112 thereby releasably locking/holding it in place. In order to return handle 112 to the closed position, the user may manually depress one of wedges 360a/b while rotating handle 112 about fastener 112a. Wedges 360a/b may be protrusions made from an elastically deformable material.

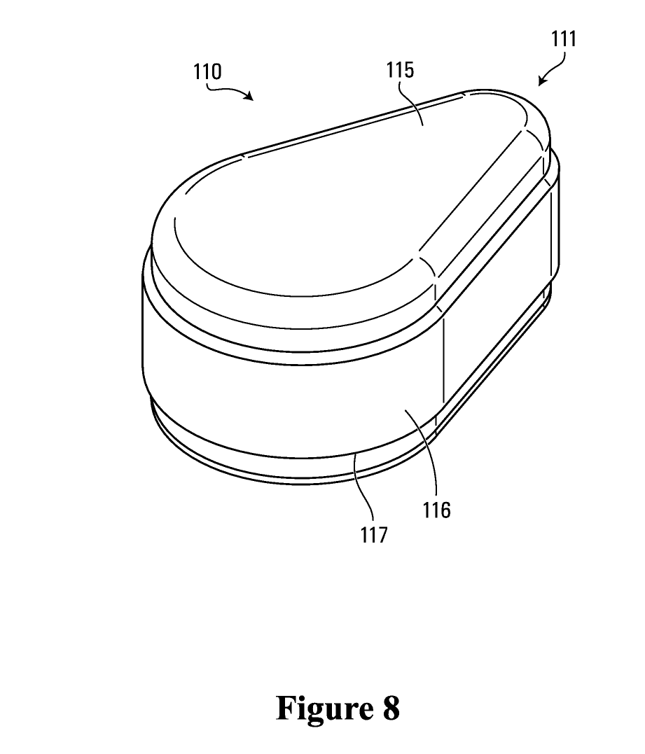

Referring to FIG. 8, there is shown an embodiment of applicator 110 which includes a cap 111. Cap 111 comprises a cap plate 115, which is shaped and dimensioned similar to base plate 153, and a sidewall 116 which is shaped and dimensioned to frictionally engage sidewall 124 and/or sidewall 158 (FIG. 2A). Sidewall 116 ends in an edge 117 which abuts base plate 153 when cap 111 is in the on position. In this position, atmospheric exposure of the internal components of applicator 110, including fluid substance 119, is substantially limited.

Applicator 110 may be configured to be refillable or to be disposable. Refillable configurations may include an access opening into first chamber 120 through which additional amounts of fluid substance 119 may be fluidly communicated into the inner cavity of first chamber 120. The access opening may be selectively opened and closed using a suitable sealing device such as an elastic deformable plug member receivable in the access opening. Refillable embodiments may include mechanisms which permit reversing the movement of press plate assembly 130 within the cavity of chamber 120 so that press plate assembly 130 can be returned to a withdrawn position to allow additional amounts of fluid substance 119 to be positioned in the inner cavity between press plate assembly 130 and screen 121/plate 122. For example, press plate assembly 130 may be readily returned to its retracted position by rotating turning wheel 160 (in the embodiment of FIG. 4B) in the opposite rotational direction to the direction that moves the press plate assembly within the inner cavity of first chamber 120 towards plate 122.

It may be appreciated that many of the plastic components of applicator 110 may be manufactured by techniques such as injection molding and 3D printing.

It will be appreciated by those skilled in the art that changes could be made to the various aspects of the subject application described above without departing from the inventive concept thereof. It is to be understood, therefore, that this subject application is not limited to the particular aspects disclosed, but it is intended to cover modifications as defined by the appended claims.

When introducing elements of the present disclosure or the embodiments thereof, the articles “a,” “an,” “the,” and “said” are intended to mean that there are one or more of the elements. The terms “comprising,” “including,” and “having” are intended to be inclusive and mean that there may be additional elements other than the listed elements.

1. A hand-held applicator for applying a fluid substance to a skin surface, said applicator comprising:
a first chamber defining at least in part a cavity for holding a fluid substance, the first chamber comprising an interface surface with a plurality of openings there through;
a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad;
a pressure increasing mechanism operable to be actuated by an operator to exert a positive pressure on the fluid substance held within the cavity to cause at least a portion of the fluid substance to move through said openings of said interface surface into said pad;
wherein the pad has a top view outer perimeter profile that is generally wedge shaped.

2. An applicator as claimed in claim 1 further comprising an upper plate that provides said interface surface and wherein said pressure increasing mechanism comprises:
a movable plate device located within said cavity of said first chamber and operable to move therein;
a movement mechanism generally positioned proximate to said first chamber and operably engaged with said moveable plate device to move said movable plate device within said cavity;
wherein said movement mechanism is operable to be activated by an operator to cause said movable plate device to move within said cavity to exert a positive pressure on said fluid substance within said cavity to cause at least a portion of the fluid substance to move through said openings of said interface surface into said pad.

3. An applicator as claimed in claim 2 wherein at least a portion of said movement mechanism is positioned within a second chamber, and wherein said second chamber is positioned proximate said first chamber and on an opposite side of said first chamber to said pad.

4. An applicator as claimed in claim 3 wherein at least a portion of said movement mechanism extends from said second chamber into said first chamber to engage with said moveable plate device to cause said movable plate device to move within said cavity to exert a positive pressure on said fluid substance within said cavity.

5. An applicator as claimed in claim 4 wherein said movement mechanism comprises a rotatable member that is operable to engage with said moveable plate device, such that in operation, rotation of said rotatable member causes said moveable plate device to move within said first chamber.

6. An applicator as claimed in claim 5 wherein said movement mechanism further comprises a rotational drive mechanism operable to rotate said rotatable member.

7. An applicator as claimed in claim 3 wherein the first chamber and the pad each have a top view outer perimeter profile that is substantially the same and generally wedge shaped.

8. An applicator as claimed in claim 3 wherein the first chamber, the second chamber and the pad have a top view perimeter profile that is substantially the same and generally wedge shaped.

9. An applicator as claimed in claim 2 wherein said movement mechanism is operable to be actuated by an operator's hand while said applicator is being held in said operator's hand.

10. An applicator as claimed in claim 2 wherein said movement mechanism comprises: (i) a rotational member interconnected to said movable plate device, operable such that rotation of said rotational member causes said movable plate device to move within said cavity of said first chamber; (ii) a gear mechanism interconnected to said rotational member operable such that rotation of said gear mechanism causes said rotational member to rotate; and (iii) a gear drive mechanism operable to be actuated by an operator to drive rotation of said gear drive mechanism;
wherein said gear drive mechanism is operable to be actuated by an operator to cause said gear mechanism to rotate said rotational member to move said movable plate device within said cavity to exert a positive pressure on said fluid substance within said cavity to cause at least a portion of the fluid substance held in said cavity to move through said openings of said interface surface into said pad.

11. An applicator as claimed in claim 10 wherein said gear drive mechanism comprises a ratchet member operable for reciprocating inward and outward translational movement relative to said second chamber, said ratchet member being operably engaged with said gear mechanism, said ratchet member being interconnected to a button operable to be actuated by an operator to cause said ratchet member to move inward and outward.

12. An applicator as claimed in claim 11 wherein said gear mechanism and said ratchet member are positioned within a second chamber positioned adjacent said first chamber and on an opposite side of said first chamber to said pad, and wherein said button extends beyond said second chamber to be capable of being operated by an operator.

13. An applicator as claimed in claim 11 further comprising an elastically deformable device operably engaged between said button and said ratchet member to provide an outwardly directed restoring force on said button when said button is moved inwards by a force exerted by an operator.

14. An applicator as claimed in claim 10 wherein:
said movable plate device has a threaded opening there through; and
said rotational member comprises a threaded member having a portion engagingly received through said threaded opening in said plate device, such that rotation of said threaded member causes said movable plate device to move within said cavity of said first chamber.

15. An applicator as claimed in claim 10 wherein:
said gear drive mechanism comprises: (a) a motor having a rotatable shaft, said shaft configured for operable engagement with said gear mechanism; (b) a source of power for driving said motor; (c) a switch operable to be actuated by an operator to selectively activate and de-activate said motor;
wherein when said motor is actuated by an operator said movable plate device moves within said cavity to exert a positive pressure on said fluid substance within said cavity to cause at least a portion of the fluid substance to move through said openings of said interface surface into said pad.

16. An applicator as claimed in claim 1 further comprising a mesh screen located within said first chamber, said mesh screen configured and located such that in operation, when said pressure increasing mechanism exerts a positive pressure on the fluid substance held within the cavity, it causes at least a portion of the fluid substance to first move through said mesh screen and then through said openings of said interface surface into said pad.

17. An applicator as claimed in claim 1 wherein said pad has a plurality of openings defined therein which may in operation be aligned with the openings in said interface surface of said first chamber such that at least a portion of said fluid substance within said cavity of said first chamber can be fluidly communicated from said first chamber through the plurality of openings of the interface surface into the openings in said pad.

18. An applicator as claimed in claim 10 wherein said movement mechanism comprises: (i) a threaded member operably interconnected to said movable plate device, such that rotation of said rotational member causes said movable plate device to move within said cavity of said first chamber; (ii) a turning wheel operably interconnected to said threaded member operable such that rotation of said turning wheel causes said threaded member to rotate; wherein said turning wheel is operable to be actuated by an operator to cause said threaded member to rotate to cause said movable plate device to move within said cavity to exert a positive pressure on said fluid substance within said cavity to cause at least a portion of the fluid substance to move through said openings of said interface surface into said pad.

19. An applicator as claimed in claim 18 wherein a portion of said turning wheel is positioned within a second chamber positioned adjacent said first chamber and on an opposite side of said first chamber to said pad, and wherein said turning wheel has a portion that extends beyond said second chamber to be capable of being operated by an operator.

20. An applicator as claimed in claim 1 further comprising a handle configured for holding by an operator, said handle being movable between an open extended position and a closed retracted position.

21. An applicator as claimed in claim 20 wherein the handle is rotatable between the open position and the closed position.

22. An applicator as claimed in claim 20 further comprising a releasable locking mechanism for releasably locking the handle in the open position or the closed position.

23. An applicator as claimed in claim 20 wherein the handle has a top view outer perimeter profile configured such that when said handle is in said closed position said outer perimeter profile of said handle is aligned with the outer perimeter profile of said first chamber.

24. An applicator as claimed in claim 7 further comprising a second chamber which is positioned proximate said first chamber and on an opposite side of said first chamber to said pad, said applicator further comprising a handle configured for holding by an operator, said handle being movable between an open position and a closed position, said handle being mounted to an outward facing surface of said second chamber.

25. An applicator as claimed in claim 24 wherein the handle is rotatable between the open position and the closed position.

26. An applicator as claimed in claim 25 further comprising a releasable locking mechanism for releasably locking the handle in the open position or the closed position.

27. An applicator as claimed in claim 24 wherein the handle has an outer perimeter profile configured such that when said handle is in said closed position said outer perimeter profile of said handle is generally aligned with the outer perimeter profile of said second chamber.

28. An applicator as claimed in claim 27 wherein the handle has an outer perimeter profile configured such that when said handle is in said closed position said outer perimeter profile of said handle is generally aligned with the outer perimeter profile of said first chamber, said second chamber and said pad.

29. A hand-held applicator for applying a fluid substance to a skin surface, said applicator comprising:
a first chamber defining at least in part a cavity for holding a fluid substance, the first chamber comprising an upper wall providing an interface surface having a plurality of openings there through;
a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad;
a force application mechanism operable to be actuated by an operator to exert a force on the fluid substance held within the cavity to cause at least a portion of the fluid substance to move through said openings of said interface surface into said pad from where an operator can transfer a portion of said fluid substance to a skin surface;
wherein the pad has first portion and a second portion, and wherein a top view outer perimeter profile of said first portion is generally wider than the top view outer perimeter profile of said second portion, said second portion being adapted for applying said fluid substance to a narrow or small area of a skin surface.

30.-44. (canceled)

45. A hand-held applicator for applying a fluid substance to a skin surface, said applicator comprising:
a first chamber defining at least in part a cavity for holding a fluid substance, the first chamber comprising an upper wall providing an interface surface with a plurality of openings;
a pad having a surface for contacting a skin surface, said pad being positioned proximate said interface surface of said first chamber and said pad being fluidly interconnected to said cavity of said first chamber such that at least a portion of said fluid substance within said cavity of said first chamber can be fluidly communicated from said first chamber through the plurality of openings of the interface surface into said pad;
a pressure increasing mechanism operable to be actuated by an operator to exert a positive pressure on the fluid substance held within the cavity to cause at least a portion of the fluid substance to move through said openings of said interface surface into said pad; said pressure increasing mechanism comprising:
(a) a movable plate device located within said cavity of said first chamber and operable to move therein, said movable plate device having a threaded opening there through;
(b) a movement mechanism positioned generally proximate to said first chamber and operably engaged with said moveable plate device to move said movable plate device within said cavity; said movement mechanism comprising: (i) a threaded member operably interconnected to said movable plate device, such that rotation of said rotational member causes said movable plate device to move within said cavity of said first chamber; (ii) a turning wheel operably interconnected to said threaded member operable such that rotation of said turning wheel causes said threaded member to rotate;
wherein said turning wheel is operable to be actuated by an operator to cause said threaded member to rotate to cause said movable plate device to move within said cavity to exert a positive pressure on said fluid substance within said cavity to cause at least a portion of the fluid substance to move through said openings of said interface surface into said pad.

46. An applicator as claimed in claim 45 wherein a portion of said turning wheel is positioned within a second chamber positioned adjacent said first chamber and on an opposite side of said first chamber to said pad, and wherein said turning wheel has a portion that extends beyond said second chamber to be capable of being operated by an operator.

47.-62. (canceled)

63. A hand-held applicator for applying a fluid substance to a skin surface, said applicator comprising:
a first chamber defining at least in part a cavity for holding a fluid substance, the first chamber comprising a wall with an interface surface and having a plurality of openings there through;
a flow restrictor plate having a plurality of openings there through, said restrictor plate being positioned adjacent to said interface surface of said wall of said first chamber;
a pad having a surface for contacting a skin surface, said pad being positioned proximate said interface surface of said wall of said first chamber and said pad being fluidly interconnected to said cavity of said first chamber such that at least a portion of said fluid substance within said cavity of said first chamber can be fluidly communicated from said first chamber through the plurality of openings of the interface surface into said pad;
a pressure increasing mechanism operable to be actuated by an operator to exert a positive pressure on the fluid substance held within the cavity to cause at least a portion of the fluid substance to move through said openings of said interface surface into said pad;
and wherein the flow restrictor plate is configured and operable for actuation by an operator to be movable between: (a) a first position in which a group comprising one or more openings of the interface surface are unblocked which permits the fluid substance to flow through the first group of openings of said interface surface into said pad, and (b) a second position in which at least some of the openings in said group of openings of the interface surface is blocked to restrict the fluid substance from flowing through any or all of the first group of openings of said interface surface into said pad.

64. An applicator as claimed in claim 63 wherein the flow restrictor plate is configured and operable for actuation by an operator to be movable between: (a) said first position in which a first group comprising one or more openings of the interface surface are unblocked which permits the fluid substance to flow through the first group of openings of said interface surface into said pad, and (b) a second position in which some of the openings in said first group of openings of the interface surface are blocked and other one or more openings in said interface surface are unblocked, so as to restrict the fluid substance from flowing through only some of the first group of openings of said interface surface into said pad.

65. An applicator as claimed in claim 64 wherein the pad has a top view perimeter profile that provides a narrow end tip portion, and wherein said second group of openings are generally in and proximate to the interface surface at the end tip portion of the first chamber and wherein in said second position of said flow restrictor plate the fluid substance may only flow through said some of the first group of openings of said interface surface into said pad in and proximate to the end tip portion of the pad.

66. An applicator as claimed in claim 63 wherein the flow restrictor plate is configured and operable for actuation by an operator to be movable between: (a) said first position in which a first group comprising one or more openings of the interface surface are unblocked which permits the fluid substance to flow through the first group of openings of said interface surface into said pad, and (b) a second position in which all of the openings in said first group of openings of the interface surface are blocked to block the fluid substance from flowing through any of the first group of openings of said interface surface into said pad.

67. An applicator as claimed in claim 66 wherein said first group of openings in said interface surface comprises all the openings in said interface surface such that in said second position all of the openings of the interface surface are blocked to block the fluid substance from flowing into said pad.

68. An applicator as claimed in claim 62 wherein said flow restrictor plate comprises a tab member that extends outwardly from said first chamber and is operable by an operator to move said flow restrictor plate between said first position and said second position.

69.-74. (canceled)

75. An applicator as claimed in claim 1 wherein the pad is made of a material selected from the group consisting of a cellulose fiber, a foamed plastic polymer, a natural rubber, a synthetic rubber and a combination thereof.

76. An applicator as claimed in claim 1 wherein the pad has a plurality of openings defined therein which are outwardly tapered to narrow in the direction of flow.

77. An applicator as claimed in claim 1 wherein the pad has a natural porosity that permits the fluid substance that flows through said openings in said interface surface to disperse throughout said pad.

78. An applicator as claimed in claim 1 wherein the pad is releasably attached to said first chamber.

79. An applicator as claimed in claim 1 further comprises a cap that is releasably attachable and is configured to cover said pad.

80. An applicator as claimed in claim 1 wherein the fluid substance is a sunscreen lotion.

81. (canceled)
